(19) United States
(12) Patent Application Publication
(10) Pub. No.: US 2023/0381205 A1
(43) Pub. Date: Nov. 30, 2023

(54) COMPOSITIONS AND METHODS FOR TREATING NEURONAL DISORDERS WITH CANNABINOIDS

(71) Applicant: INMED PHARMACEUTICALS INC., Coquitlam (CA)

(72) Inventors: Eric HSU, Vancouver (CA); Salam KADHIM, Coquitlam (CA); Rishi SOMVANSHI, Vancouver (CA); Ujendra KUMAR, Richmond (CA)

(21) Appl. No.: 18/033,187

(22) PCT Filed: Oct. 21, 2021
(86) PCT No.: PCT/CA21/51487

§ 371 Date: Apr. 21, 2023

Related U.S. Application Data
(60) Provisional application No. 63/094,822, filed on Oct. 21, 2020.

(51) Int. Cl. A61K 31/00; A61P 25/28

(52) U.S. Cl. CPC A61K 31/658; A61P 25/28

(57) ABSTRACT

Provided herein are methods and compositions comprising a cannabinoid compound for providing neuroprotection and/or stimulating neuritogenesis. The cannabinoid compound can be a compound of Formula (I), wherein R¹ is COOH or H, R² is C₃H₇ or C₅H₁₁, R³ is H or Me, R⁴ and R⁵ are Me or (CH₂)₂CH═C(CH₃)₂, such as CBGA, a derivative thereof, a prodrug thereof, or combinations thereof, and can be used in the treatment of neurodegenerative diseases, or to promote neurite elongation and/or restore neurite formation in patients in need thereof.

### RELATED APPLICATIONS

This application claims the benefit of priority to U.S. Provisional Patent Application Ser. No. 63/094,822, filed Oct. 21, 2020, which is herein incorporated by reference in its entirety.

### BACKGROUND OF THE INVENTION

Some 100 million Americans suffer from neurological and brain disorders at some point in their lives. Such diseases are often devastating, coming with a high physical, emotional, and economic cost. Indeed, according to the United States Center for Disease Control and Prevention (CDC), in 2010, the costs of treating Alzheimer's disease alone were estimated to fall between $159 billion and $215 billion. By 2040, these costs are projected to jump to between $379 billion and $500 billion annually. Unfortunately, currently available treatments for neurodegenerative diseases may relieve some of the associated symptoms, but there are no known cures.

Neurodegenerative diseases typically involve neuronal atrophy, axonal degeneration (e.g., Wallerian and/or Wallerian-like degeneration), demyelination of axons, and necrotic or programmed cell death. Different types of programmed cell death, such as apoptosis, autophagy, pyroptosis, and oncosis have all been demonstrated in neurons. Accordingly, methods and compositions that block or reverse these processes and thus promote neuroprotection, would effectively prevent, reverse, or delay some or all neurodegenerative symptoms.

Cannabis plants produce many compounds, including cannabinoids, some of which may be of medical importance. Indeed, some cannabis plant extracts have shown some beneficial effects in treating brain injury (see e.g., U.S. Pat. No. 9,205,063). In addition, there are many anecdotal reports of potential therapeutic effects. However, many cannabinoids and their derivatives exhibit no detectable neuroprotective effect at physiological concentrations, and others have been shown to contribute to excitotoxicity at physiological concentrations. In cases where cannabinoids may have documented effects, the therapeutic potential of cannabinoids in Alzheimer's disease and other ailments is largely attributed to the effects of THC and CBD and other cannabinoid compounds (see e.g., US 2017/0273914, US 2018/0169035). Indeed, THC and CBD have been proposed to act as free-radical scavenging antioxidants and neuroprotectants (see e.g., U.S. Pat. No. 6,630,507).

Given the many symptoms and presentations of neurodegenerative disease, there remains a need in the art for improved methods and compositions for treating neurodegenerative diseases with cannabinoid compounds. Fortunately, as will be clear from the detailed description that follows, the present disclosure provides for these and other needs.

### SUMMARY OF THE INVENTION

Described herein are cannabinoid compounds, pharmaceutical compositions comprising the same and methods of use in treating neuronal disorders, including those characterized by neurodegeneration, and/or those requiring or benefiting from neuritogenesis. Without being bound by theory, the particular cannabinoid compounds disclosed herein may inhibit or slow the progression of a neurodegenerative disease by reducing cytotoxicity in a population of affected neurons. Additionally, and remarkably, the subject cannabinoid compounds can also be used to promote neurite elongation and/or restore neurite formation in damaged neurons, and in patients in need thereof.

In one aspect, methods of treating a patient with a neuronal disorder characterized by neurodegeneration are provided, comprising administering a therapeutically effective amount of a pharmaceutical composition comprising a cannabinoid compound to a patient in need thereof, wherein the cannabinoid compound inhibits or slows the progression of the neurode generative disease. In embodiments, the cannabinoid compound is a compound of any one of Formulas I-VIIA and A-F. In embodiments, the cannabinoid compound is a compound of Formula I. In an exemplary and preferred embodiment, the cannabinoid compound is cannabigerolic acid (CBGA) or a derivative thereof, prodrugs thereof, or combinations thereof.

In one aspect, methods of treating a patient with a neuronal disorder benefitting from neuritogenesis are provided, comprising administering a therapeutically effective amount of a pharmaceutical composition comprising a cannabinoid compound to a patient in need thereof, wherein the cannabinoid compound stimulates neuritogenesis. In embodiments, the cannabinoid compound is a compound of any one of Formulas I-VIIA and A-F. In embodiments, the cannabinoid compound is a compound of Formula I. In an exemplary and preferred embodiment, the cannabinoid compound is CBGA or a derivative thereof, prodrugs thereof, or combinations thereof.

In embodiments, the neuronal disorder is a Central Nervous System (CNS) or a Peripheral Nervous System (PNS) disorder. In embodiments, the CNS disorder is selected from the group comprising or consisting of Alzheimer's Disease (AD), Parkinson's Disease (PD), Amyotrophic Lateral Sclerosis (ALS), Huntington's Disease (HD) and Multiple Sclerosis (MS). In an exemplary embodiment, the CNS disorder is AD. In embodiments, the PNS disorder is selected from the group comprising or consisting of entrapment neuropathy (e.g. carpal tunnel syndrome); thoracic outlet syndrome, brachial plexus injury (e.g., as seen in a motorcycle upper extremity traction injury); direct open traumatic injury, diabetic nerve problems, Guillain-Barre syndrome, hereditary sensory and autonomic neuropathies (HSANs) (e.g. familial dysautonomia). The cannabinoid compounds and pharmaceutical compositions disclosed herein can be locally or systemically administered to a subject to inhibit or slow neurodegenerative disease progression and/or to stimulate neuritogenesis in the subject. In embodiments, a disorder benefitting from neuritogenesis is selected from the group comprising or consisting of axonal injury, ischemic stroke, schizophrenia, Down syndrome, autism spectrum disorder (ASD), amyotrophic lateral sclerosis (ALS), primary lateral sclerosis, spinal muscular atrophy, motor neuron diseases, chronic hearing loss, tinnitus, hyperacusis, presbycusis, and balance disorders associated with cochlear synaptopathy and vestibular synaptopathy. In further embodiments, the methods further comprises the simultaneous or sequential administration of one or more additional active agent(s).

In another aspect, methods of promoting neurite elongation and/or restoring neurite formation are provided for patients in need thereof, comprising administering to the patient a therapeutically effective amount of a cannabinoid compound as described herein. In embodiments, the cannabinoid compound is a compound of any one of Formulas I-VIIA and A-F. In embodiments, the cannabinoid compound is a compound of Formula I, or a derivative thereof, or combinations thereof. In an exemplary and preferred embodiment, the cannabinoid compound is CBGA or a derivative thereof, prodrugs thereof, or combinations thereof. Patients in need of neurite elongation and/or restoration of neurite formation may include, e.g., those patients suffering from axonal injury, ischemic stroke, schizophrenia, Down syndrome, autism spectrum disorder (ASD), amyotrophic lateral sclerosis (ALS), primary lateral sclerosis, spinal muscular atrophy, motor neuron diseases, chronic hearing loss, tinnitus, hyperacusis, presbycusis, and balance disorders associated with cochlear synaptopathy and vestibular synaptopathy.

Embodiments include the use of a cannabinoid compound of any one of Formulas I-VIIA and A-F for treating neurodegeneration in a patient in need thereof. Embodiments include the use of a cannabinoid compound of Formula I, a derivative thereof, or combinations thereof for treating neurodegeneration in a patient in need thereof. Embodiments include the use of a cannabinoid compound of any one of Formulas I-VIIA and A-F for promoting neurite elongation and/or restoring neurite formation in a patient in need thereof. Embodiments also include the use of a cannabinoid compound of Formula I, a derivative thereof, or combinations thereof, for promoting neurite elongation and/or restoring neurite formation in a patient in need thereof.

In embodiments, the cannabinoid compound and pharmaceutical compositions comprising same can be administered by intracerebroventricular (i.c.v.) injection, which may be weekly, daily or twice daily.

In embodiments, the cannabinoid compound and pharmaceutical compositions comprising same can be administered systemically, e.g. intravenously. In embodiments, systemic administration comprises transdermal administration. In embodiments, the pharmaceutical composition comprising the cannabinoid compound is administered orally, e.g. as a pill, an extended release capsule or a sublingual spray or film.

In embodiments, the pharmaceutical composition comprising the cannabinoid compound is administered locally. In another embodiment, the pharmaceutical composition comprising the cannabinoid compound is administered directly to the brain.

In another aspect, pharmaceutical compositions are provided comprising the cannabinoid compound of Formula I, a derivative thereof, or combinations thereof. In embodiments, the cannabinoid compound is a compound of any one of Formulas I-VIIA and A-F. In embodiments, the cannabinoid compound is CBGA or a derivative thereof, prodrugs thereof, or combinations thereof. The pharmaceutical composition may be an injectable formulation, an injectable microemulsion or nanoparticle formulation, an intravenous formulation, an intranasal spray, a sublingual spray or film, or an oral formulation.

In exemplary embodiments, the compound of any one of Formulas I-VIIA and A-F is provided in an extended-release formulation where it can be applied locally for peripheral nerve disorders. In exemplary embodiments, CBGA or a derivative thereof, a prodrug thereof, or a combination thereof is provided in an extended-release formulation where it can be applied locally for peripheral nerve disorders. In embodiments, the compound of any one of Formulas I-VIIA and A-F is provided in an injectable microemulsion or nanoparticle formulation. In embodiments, CBGA or a derivative thereof, a prodrug thereof, or a combination thereof is provided in an injectable microemulsion or nanoparticle formulation.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

FIG. 1 Pre-screen of Cannabinoids. FIG. 1 illustrates in vitro neuroprotection of differentiated SH-SY5Y human neuronal cells by contacting the cells with CBD, CBDA, CBC, CBG, CBGA, CBN, CBND, Δ9-THC at 0.5 μM, 1.5 μM, 5 μM, 15 μM respectively, for each cannabinoid. Vehicle Control (VC) contained 0.15% Ethanol. Data presented as Cell Death (%) vs Vehicle Control (taken as 0%). As shown in the figure, CBD, CBDA, CBC, CBG, CBN, CBND and Δ9-THC promote cell death of SH-SY5Y human neuronal cells at 15 μM. In contrast, CBGA is pro-survival.

FIG. 2 STSR-induces significant dose-dependent cytotoxicity of differentiated SH-SY5Y neuronal cells. FIG. 2 illustrates in vitro insult-induced cytotoxicity of Staurosporin (STSR) on differentiated SH-SY5Y human neuronal cells at 75 nM, 100 nM, 150 nM, 200 nM concentrations. Briefly, 15,000 cells/well were plated and differentiated with Retinoic Acid (RA) for 3 days. Cells were treated with Staurosporin in a dose dependent manner for 72 hours (hrs) and processed for MTT assay. Data presented as Cell Death (%) vs Vehicle Control (taken as 0%). Statistically significant cytotoxicity for STSR at all concentrations compared to VC (p<0.0001) by One-Way analysis of variance (ANOVA). One-way ANOVA is known in the art (see e.g., Howell, David (2002) Statistical Methods for Psychology. Duxbury. pp. 324-325).

FIG. 3 CBGA confers dose-dependent neuroprotection to differentiated SH-SY5Y cells from STSR (75 nM) induced cytotoxicity. CBN, CBND and CBD treatment with STSR under the same conditions results in cell death. FIG. 3 illustrates neuroprotective/cytotoxic effects of CBGA, CBN, CBND and CBD at 0.5 μM, 1.5 μM and 5 μM concentrations on differentiated SH-SY5Y human neuronal cells when cultured concurrently with STSR at 75 nM. Briefly, 15,000 cells/well were plated and differentiated with RA for 3 days. Cells were treated with Cannabinoids along with Staurosporin (75 nM) in a dose dependent manner for 72 hrs and processed for MTT assay. Data presented as Cell Death (%) vs Staurosporin (STSR) at 75 nM (taken as 0%). Vehicle Control (VC): DMSO and Ethanol. Statistically significant neuroprotection for STSR+CBGA (p=0.001) at 5 μM compared to STSR-alone and statistically significant cytotoxicity for STSR+CBN, STSR+CBND, STSR+CBD (p<0.0001) at 5 μM compared to STSR-alone by One-Way ANOVA.

FIG. 4 CBGA treatment of differentiated SH-SY5Y cells is not toxic at high concentrations under basal conditions. FIG. 4 illustrates the lack of in vitro cytotoxicity by CBGA on differentiated SH-SY5Y human neuronal cells at 1.5 μM, 5 μM, 10 μM, 15 μM, 20 μM concentrations under basal conditions. Data for CBGA is presented as Cell Death (%) vs Vehicle Control (taken as 0%).

FIG. 5 CBGA confers neuroprotection to differentiated SH-SY5Y cells from STSR (75 nM) induced cytotoxicity. FIG. 5 illustrates the neuroprotective effect of CBGA at 0.5 μM, 1.5 μM, 5 μM, 10 μM, 15 μM, 20 μM on differentiated SH-SY5Y human neuronal cells from STSR (75 nM) insult-induced cytotoxicity. Data for STSR+CBGA is presented as Cell Death (%) vs Staurosporin (STSR) (taken as 0%). Statistically significant cytotoxicity for STSR at 75 nM (p=0.0001) compared to VC and for STSR+CBGA at 10 μM, 15 μM and 20 μM (p<0.05-p≤0.0001) compared to STSR-alone by One-Way ANOVA.

FIG. 6 illustrates the comparative neuroprotective effect of CBGA versus CBG in the presence of STSR (75 nM) induced cytotoxic insult on differentiated SH-SY5Y human neuronal cells at 5 μM, 10 μM, 15 μM concentrations. CBGA at 5, 10 and 15 μM confers neuroprotection to differentiated SH-SY5Y cells from STSR (75 nM) induced cytotoxicity. CBG treatment of cells with STSR at the same concentrations and conditions results in cell death. Data for STSR+CBGA and STSR+CBG is presented as Cell Death (%) vs Staurosporin (STSR) (taken as 0%). Statistically significant neuroprotection for STSR+CBGA at 10 μM and 15 μM (p<0.05-p≤0.01) compared to STSR at 75 nM and statistically significant cytotoxicity for STSR+CBG at 15 μM (p=0.0002) compared to STSR at 75 nM by One-Way ANOVA.

FIG. 7 illustrates the comparative neuroprotective effect of CBGA versus CBG in the presence of Aβ1-42 (5 μM) induced cytotoxic insult on differentiated SH-SY5Y human neuronal cells for CBGA at 0.15 μM, 0.5 μM, 1.5 μM, 10 μM, 15 μM, 20 μM and for CBG at 1.5 μM, 5 μM, 10 μM concentrations. CBGA at 5 μM, 10 μM, and 15 μM confers significant neuroprotection to differentiated SH-SY5Y cells from Aβ1-42 induced cytotoxicity. CBG treatment with Aβ1-42 at the same concentrations and conditions results in cell death. Data for Aβ1-42+CBGA and Aβ1-42+CBG is presented as Cell Death (%) vs API-42 peptide (taken as 0%). Statistically significant neuroprotection for Aβ1-42+CBGA (p<0.0001) at 5 μM, 10 μM and 15 μM compared to Aβ1-42 at 5 μM and statistically significant cytotoxicity for Aβ1-42+CBG (p<0.0001) at 10 μM compared to Aβ1-42 at 5 μM by One-Way ANOVA.

FIG. 8 illustrates the effect of a CB2 antagonist has on the reversal of CBGA mediated protection of Aβ toxicity. The CB2 antagonist SR114528 has a more prominent effect on the reversal of CBGA mediated protection. At the lowest concentration tested (1.5 μM), the CB2 antagonist completely reversed the CBGA mediated neuroprotection of the differentiated SH SY5Y cells treated with Aβ1-42. CB2 antagonist has significant effect on the reversal of CBGA mediated protection at lower concentration. p<0.0001 for Aβ compared to vehicle control. p<0.0001 for all groups compared to CBGA (10 mM)+Aβ.

FIG. 9 illustrates the upregulation of BAX (Pro-apoptotic marker) on differentiated SH SY5Y treated cells with 5 μM Aβ1-42. As shown in the figure, Aβ1-42 treatment of differentiated SH-SY5Y cells induced upregulation of BAX. CBGA treatment alone or with Aβ1-42 at 10 μM concentration restores BAX levels back to the control levels.

FIG. 10 illustrates the downregulation of phosphorylated BCL2 (anti-apoptotic marker) on differentiated SH-SY5Y treated with 5 μM Aβ1-42. CBGA treatment alone or in the presence of Aβ1-42 results in upregulated pBCL2 expression to control levels, supporting an anti-apoptotic role of CBGA.

FIG. 11 Changes in receptor expression. FIG. 11 illustrates that CBGA (5 μM, 10 μM) promotes translocation of CB1R to the Golgi apparatus in a dose-dependent manner. CBN and THC did not alter the expression of CB1R at the membrane or intracellularly.

FIG. 12 provides representative photomicrographs illustrating CBGA mediated changes in neurite elongation and branching in differentiated SH-SY5Y cells in a dose dependent manner. Cells displayed morphological changes in shape and size of cells along with neurite outgrowth. FIG. 12 also provides a histogram showing gradual changes in neurite length following treatment with CBGA. Note that CBGA in a dose dependent manner significantly increased the neuritogenesis in SH-SY5Y cells. p<0.01-p<0.0001 vs control by One Way ANOVA.

FIG. 13 CBGA upregulates the expression of Tuj1 neuritogenesis marker. Developmental changes in prominent intracellular marker of neurite growth, neuron-specific III β-tubulin (Tuj1) was determined using Western blot analysis. CBGA increased the expression of Tuj1 in differentiated SH-SY5Y cells. FIG. 13 also provides a histogram showing densitometric analysis of relative expression of Tuj1. β-Actin was used as a loading control. P<0.05 for CBGA 10 μM vs Control by One-Way ANOVA.

FIG. 14 Morphological characterization of CBGA, THC and CBN mediated changes in differentiated SH-SY5Y cells. SH-SY5Y cells were differentiated with RA (10 μM) for 5 days as indicated. (A) Representative confocal photomicrographs showing changes in the cellular distribution of Tuj1 in RA induced differentiated SH-SY5Y cells following treatment with CBGA, THC and CBN. (B) Histogram showing the effect of CBGA, CBN, THC on neurite length. Note that CBGA increased neurite length significantly in dose dependent manner. THC and CBN treatment significantly decreased the neurite length 24 hours post treatment.

FIG. 15 CBGA upregulates the expression of MAP2 neuritogenesis marker. CBGA (10 μM) increased the expression of MAP2 in differentiated SH-SY5Y cells and abrogates Aβ mediated downregulaion of MAP2 expression. Changes in prominent intracellular marker of neurite growth, microtubule-associated protein 2 (MAP2) was determined using western blot analysis. Histogram showing densitometric analysis of relative expression of MAP2. β-Actin was used as a loading control.

FIG. 16 Morphological characterization of CBGA mediated changes in differentiated SH-SY5Y cells. SH-SY5Y cells were differentiated with RA (10 μM) for 5 days as indicated. Representative confocal photomicrographs showing changes in the cellular distribution of MAP2 in RA induced differentiated SH-SY5Y cells following treatment with CBGA in the presence or absence of A (5 RM). Cells were subjected to treatment with CBGA (10 μM), Aβ1-42 (5 μM), and A β1-42 plus CBGA. Note that Aβ (5 μM) decreased and CBGA up-regulated the expression of MAP2. CBGA abrogates the down-regulation MAP2 expression as induced by Aβ (5 μM) treatment, thus restoring MAP2 expression and neurite formation.

### DETAILED DESCRIPTION OF THE INVENTION

### Definitions

As used herein, “a patient in need thereof,” and the like, refers to a mammal, preferably a human.

As used herein and unless specified otherwise (i.e. as specified otherwise for a compound of Formula VII), “alkyl” means a linear or branched hydrocarbon group having one to twenty carbon atoms. In some embodiments, alkyl has one to twelve carbon atoms. In some embodiments, alkyl has one to six carbon atoms. In some embodiments, alkyl is methyl, ethyl, propyl, isopropyl, butyl, s-butyl, t-butyl, isobutyl, pentyl, hexyl and the like. “C₆ alkyl” refers to, for example, n-hexyl, iso-hexyl, and the like.

“Alkylamine” as used herein means an —NHR group where R is alkyl, as defined in this section of the Definitions.

“Alkoxy,” as used herein, means an —OR group, where R is an alkyl group, as defined in this section of the Definitions.

“Alkoxyalkyl,” as used herein, means an alkyl group, as defined in this section of the Definitions, substituted with one or two alkoxy groups, as defined herein.

“Hydroxyalkyl,” as used herein, means an alkyl, as defined in this section of the Definitions, substituted with one or two hydroxy groups.

“Hydroxyalkylamine,” means an —NHR group where R is hydroxylalkyl, as defined in this section of the Definitions.

As used herein, “Cannabigerolic Acid” or “CBGA” refers to 3-[(2E)-3,7-dimethylocta-2,6-dienyl]-2,4-dihydroxy-6-pentylbenzoic acid. “Salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic.

Additional information on suitable pharmaceutically acceptable salts can be found in Remington's Pharmaceutical Sciences, 17th ed., Mack Publishing Company, Easton, Pa., 1985, which is incorporated herein by reference.

“Salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

As used herein, the term “solvate” means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate.” Examples of hydrate include, but are not limited to, hemihydrate, monohydrate, dihydrate, trihydrate, hexahydrate, and other water-containing species. It should be understood by one of ordinary skill in the art that the pharmaceutically acceptable salt, and/or prodrug of a compound may also exist in a solvate form. The solvate is typically formed via hydration which is either part of the preparation of a compound or through natural absorption of moisture by an anhydrous compound of the present invention. In general, all physical forms are intended to be within the scope of the present invention.

Thus, when a pharmacologically active agent included in a composition according to the present invention, such as, but not limited to, the compound of any one of Formulas I-VIIA and A-F or a derivative thereof, possesses a sufficiently acidic, a sufficiently basic, or both a sufficiently acidic and a sufficiently basic functional group, this group or groups can accordingly react with any of a number of inorganic or organic bases, and inorganic and organic acids, to form a pharmaceutically acceptable salt. Exemplary pharmaceutically acceptable salts include those salts prepared by reaction of the pharmacologically active compound with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, β-hydroxybutyrates, glycolates, tartrates, methane-sulfonates, propanesulfonates, naphthalene-1-sulfonates, naphthalene-2-sulfonates, and mandelates.

If the pharmacologically active compound has one or more basic functional groups, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, or with a pyranosidyl acid, such as glucuronic acid or galacturonic acid, or with an alpha-hydroxy acid, such as citric acid, tartaric acid, or with an amino acid, such as aspartic acid, glutamic acid, or with an aromatic acid, such as benzoic acid, cinnamic acid, or with a sulfonic acid, such as p-toluenesulfonic acid or ethanesulfonic acid, or the like.

If the pharmacologically active compound has one or more acidic functional groups, the desired pharmaceutically acceptable salt may be prepared by any suitable method available in the art, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

“Composition” as used herein is intended to encompass a product comprising the specified ingredients in the specified amounts, as well as any product that results from combination of the specified ingredients in the specified amounts. By “pharmaceutically acceptable” it is meant the carrier, diluent or excipient must be compatible with the other ingredients of the formulation and not deleterious to the recipient thereof.

“Pharmaceutically acceptable excipient” refers to a substance that aids the administration of an active agent to the subject and/or absorption by a subject. Pharmaceutical excipients useful in the present invention include, but are not limited to binders, fillers, disintegrants, lubricants, coatings, sweeteners, flavors and colors. One of skill in the art will recognize that other pharmaceutical excipients are useful in the present invention.

As used herein, the terms “therapeutically effective quantity,” “therapeutically effective dose,” or “therapeutically effective amount” refer to a dose of one or more compositions described herein that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

### Definitions for the Cannabinoid Compound as Described in WO2020/092923 (PCT/US2019/059237), e.g. a Compound Accordingly to Formulas VII and VIIA (as Numbered Herein)

Unless otherwise defined, all terms of art, notations and other scientific terminology used herein are intended to have the meanings commonly understood by those of ordinary skill in the art to which the present application pertains. In some cases, terms with commonly understood meanings are defined herein for clarity and/or for ready reference, and the inclusion of such definitions herein should not necessarily be construed to represent a substantial difference over what is generally understood in the art.

The term “alkyl,” by itself or as part of another substituent, refers to a straight or branched, saturated, aliphatic radical. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc.

The term “alkenyl,” by itself or as part of another substituent, refers to an alkyl group, as defined herein, having one or more carbon-carbon double bonds. Examples of alkenyl groups include, but are not limited to, vinyl (i.e., ethenyl), crotyl (i.e., but-2-en-1-yl), penta-1,3-dien-1-yl, and the like. Alkenyl moieties may be further substituted, e.g., with aryl substituents (such as phenyl or hydroxyphenyl, in the case of 4-hydroxystyryl).

The terms “halogen” and “halo,” by themselves or as part of another substituent, refer to a fluorine, chlorine, bromine, or iodine atom.

The term “haloalkyl,” by itself or as part of another substituent, refers to an alkyl group where some or all of the hydrogen atoms are replaced with halogen atoms. As for alkyl groups, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

The term “hydroxyalkyl,” by itself or as part of another substituent, refers to an alkyl group where some or all of the hydrogen atoms are replaced with hydroxyl groups (i.e., —OH groups). As for alkyl and haloalkyl groups, hydroxyalkyl groups can have any suitable number of carbon atoms, such as C₁₋₆.

The term “deuterated” refers to a substituent (e.g., an alkyl group) having one or more deuterium atoms (i.e., ²H atoms) in place of one or more hydrogen atoms.

The term “tritiated” refers to a substituent (e.g., an alkyl group) having one or more tritium atoms (i.e., ³H atoms) in place of one or more hydrogen atoms.

The term “prenyl moiety” refers to a substituent containing at least one methylbutenyl group (e.g., a 3-methylbut-2-ene-1-yl group). In many instances prenyl moieties are synthesized biochemically from isopentenyl pyrophosphate and/or isopentenyl diphosphate, giving rise to terpene natural products and other compounds. Examples of prenyl moieties include, but are not limited to, prenyl (i.e., 3-methylbut-2-ene-1-yl), isoprenyl (i.e., 3-methylbut-3-ene-1-yl), geranyl, myrcenyl, ocimenyl, farnesyl, and geranylgeranyl.

### Cannabinoid Compounds

Cannabinoids are a group of chemicals known to activate cannabinoid receptors in cells throughout the human body, including the skin. Phytocannabinoids are the cannabinoids derived from cannabis plants. They can be isolated from plants or produced synthetically. Endocannabinoids are endogenous cannabinoids produced naturally by cells in the human body. Canonical phytocannabinoids are tricyclic terpenoid compounds bearing a benzopyran moiety.

Cannabinoids include, but are not limited to, phytocannabinoids. In some cases the cannabinoids include, but are not limited to, cannabinol (CBN), cannabidiol (CBD), A⁹-tetrahydrocannabinol (Δ⁹-THC), the synthetic cannabinoid HU-210 (6aR,10aR)-9-(hydroxymethyl)-6,6-dimethyl-3-(2-methyloctan-2-yl)-6H,6aH,7H,10H,10aH-benzo[c]isochromen-1-ol), HU-308 ([(1R,2R,5R)-2-[2,6-dimethoxy-4-(2-methyloctan-2-yl)phenyl]-7,7-dimethyl-4-bicyclo[3.1.1]hept-3-enyl]methanol), HU-433 an enantiomer of HU-308, cannabidivarin (CBDV), cannabichromene (CBC), cannabichromevarin (CBCV), cannabigerol (CBG), cannabigerolic acid (CBGA), cannabigerovarin (CBGV), cannabielsoin (CBE),cannabicyclol (CBL), cannabivarin (CBV), and cannabitriol (CBT). Still other cannabinoids include, including tetrahydrocannibivarin (THCV) and cannabigerol monomethyl ether (CBGM). Additional cannabinoids include cannabichromenic acid (CBCA), Δ⁹-tetrahydrocannabinolic acid (THCA); and cannabidiolic acid (CBDA); these additional cannabinoids are characterized by the presence of a carboxylic acid group in their structure.

Still other cannabinoids include nabilone, rimonabant, JWH-018 (naphthalen-1-yl-(1-pentylindol-3-yl)methanone), JWH-073 naphthalen-1-yl-(1-butylindol-3-yl)methanone, CP-55940 (2-[(1R,2R,5R)-5-hydroxy-2-(3-hydroxypropyl) cyclohexyl]-5-(2-methyloctan-2-yl)phenol), dimethylheptylpyran, HU-331 (3-hydroxy-2-[(1R)-6-isopropenyl-3-methyl-cyclohex-2-en-1-yl]-5-pentyl-1,4-benzoquinone), SR144528 (5-(4-chloro-3-methylphenyl)-1-[(4-methylphenyl)methyl]-N-[(1S,2S,4R)-1,3,3-trimethylbicyclo[2.2.1]heptan-2-yl]-1H-pyrazole-3-carboxamide), WIN 55,212-2 ((11R)-2-methyl-11-[(morpholin-4-yl)methyl]-3-(naphthalene-1-carbonyl)-9-oxa-1-azatricyclo[6.3.1.0^{4,12}]dodeca-2,4(12),5,7-tetraene), JWH-133 ((6aR,10aR)-3-(1,1-dimethylbutyl)-6a,7,10,10a-tetrahydro-6,6,9-trimethyl-6H-dibenzo[b,d]pyran), levonatradol, and AM-2201 (1-[(5-fluoropentyl)-1H-indol-3-yl]-(naphthalen-1-yl)methanone). Other cannabinoids include Δ⁸-tetrahydrocannabinol (Δ⁸-THC), 11-hydroxy-Δ⁹-tetrahydrocannabinol, Δ¹¹-tetrahydrocannabinol, and 11-hydroxy-tetracannabinol.

Cannabinoids exert their effects by interacting with cannabinoid receptors present on the surface of the cells. To date, two types of cannabinoid receptors have been identified, the CB1 receptor and the CB2 receptor. These two receptors share about 48% amino acid sequence identity and are distributed in different tissues, and have distinct cell signaling mechanisms. They also differ in their sensitivity to agonists and antagonists, and the myriad cannabinoids exert myriad impacts on one or sometimes both receptors, making functional generalizations very difficult. Notably, it was recently shown that certain cannabinoid-based agonists of the CB2 receptor may actually induce neuronal damage, Wojcieszak et al., J Mol Neurosci (2016) 58:441-445, and as such determining the appropriate type and/or level of cannabinoid receptor interaction in neuronal tissues has thus far proven elusive.

Without being bound by theory, and contrary to the findings of Wojcieszak et al. above, the preferred cannabinoid compounds according to the present invention may selectively bind the CB2 cannabinoid receptor and act as a partial or full agonist. In embodiments, the cannabinoid compounds of the subject invention may bind to both the CB1 and CB2 receptors, but exhibit a higher affinity to the CB2 receptor. In embodiments, the cannabinoid compounds of the subject invention may down-regulate the expression of the CB1 receptor, and/or modulate the translocation of CB1R to the membrane of the neuron. In embodiments, the cannabinoid compounds of the subject invention may up-regulate the expression of the CB2 receptor, and/or increase the translocation of CB2R to the membrane. In some embodiments, the cannabinoid compound is according to Formula (I).

Without being bound by theory, the preferred cannabinoid compounds according to the present invention may increase neurite outgrowth, and/or enhance expression of neuronal microtubule-associated protein (MAP2) in the cells and neurites, and thereby provide stability to neuronal cells. In embodiments, the cannabinoid compounds of the subject invention may enhance the expression of the building blocks of microtubules, e.g. the Tuj1 protein, and thereby stabilize axonal structures and dendrites to improve neuronal communication.

Certain structures in this disclosure include a double bond. In such structures, the double bond may be in the E- or Z-configuration (regardless of how the double bond is drawn), unless context provides otherwise. In a preferred embodiment, the double bond is in the E-configuration.

In embodiments, the cannabinoid compound is according to Formula I:

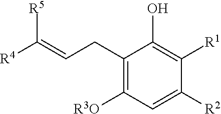

wherein
R¹ is COOH, R² is n-C₅H₁₁, R³ is H, R⁴ is (CH₂)₂CH═C(CH₃)₂ and R⁵ is Me;
R¹ is COOH, R² is n-C₅H₁₁, R³ is Me, R⁴ is (CH₂)₂CH═C(CH₃)₂ and R⁵ is Me;
R¹ is COOH, R² is n-C₃H₇, R³ is H, R⁴ is (CH₂)₂CH═C(CH₃)₂ and R⁵ is Me;
R¹ is H, R² is n-C₃H₇, R³ is H, R⁴ is (CH₂)₂CH═C(CH₃)₂ and R⁵ is Me; or
R¹ is COOH, R² is n-C₅H₁₁, R³ is H, R⁴ is Me and R⁵ is (CH₂)₂CH═C(CH₃)₂;
   or a derivative thereof.

In embodiments, the cannabinoid compound is cannabinogerolic acid or a derivative compound according to Formula A:

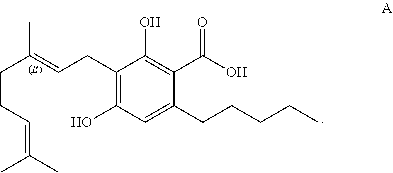 [SMILES: CCCCCc1cc(O)c(C/C=C(\C)CCC=C(C)C)c(O)c1C(=O)O]

In embodiments, the cannabinoid compound can be a CBGA-derivative such as CBGAM (Cannabigerolic acid A monomethyl ether) [(E)-CBGAM-C5 A (cis)] according to Formula B:

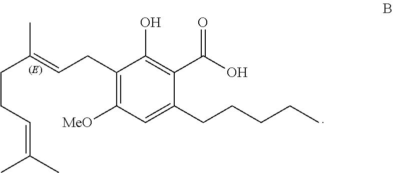 [SMILES: CCCCCc1cc(OC)c(C/C=C(\C)CCC=C(C)C)c(O)c1C(=O)O]

In embodiments, the cannabinoid compound can be a CBGA-derivative such as Cannabigerol monomethyl ether [(E)-CBGM-C5 (cis)] according to Formula C:

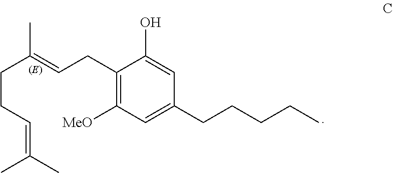 [SMILES: CCCCCc1cc(O)c(C/C=C(\C)CCC=C(C)C)c(OC)c1]

In embodiments, the cannabinoid compound can be a CBGA-derivative such as Cannabigerovarinic acid A [(E)-CBGVA-C3 A (cis)] according to Formula D:

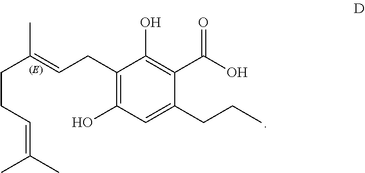 [SMILES: CCCc1cc(O)c(C/C=C(\C)CCC=C(C)C)c(O)c1C(=O)O]

In embodiments, the cannabinoid compound can be a CBGA-derivative such as Cannabigerovarin [(E)-CBGV-C3 (cis)] according to Formula E:

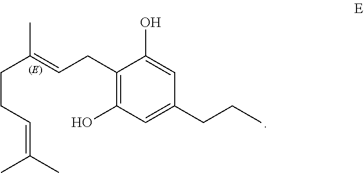 [SMILES: CCCc1cc(O)c(C/C=C(\C)CCC=C(C)C)c(O)c1]

In embodiments, the cannabinoid compound can be a CBGA-derivative such as Cannabinerolic acid A [(Z)-CBGA-C5 A (trans)] according to Formula F:

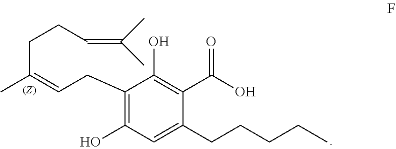 [SMILES: CCCCCc1cc(O)c(C/C=C(/C)CCC=C(C)C)c(O)c1C(=O)O]

An exemplary and preferred cannabinoid compound is CBGA.

In some cases, the cannabinoids or precursors thereof, can be purified, derivatized (e.g., to form a prodrug, solvate, or salt, or to form a target cannabinoid from the precursor), and/or formulated in a pharmaceutical composition.

As used herein, the term “prodrug” refers to a derivative that is a precursor compound that, following administration, releases the biologically active compound in vivo via some chemical or physiological process (e.g., a prodrug on reaching physiological pH or through enzyme action is converted to the biologically active compound). A prodrug itself may either lack or possess the desired biological activity. Thus, the term “prodrug” refers to a precursor of a biologically active compound that is pharmaceutically acceptable. In certain cases, a prodrug has improved physical and/or delivery properties over a parent compound from which the prodrug has been derived. The prodrug often offers advantages of solubility, tissue compatibility, or delayed release in a mammalian organism (H. Bundgard, Design of Prodrugs (Elsevier, Amsterdam, 1988), pp. 7-9, 21-24). A discussion of prodrugs is provided in T. Higuchi et al., “Pro-Drugs as Novel Delivery Systems,” ACS Symposium Series, Vol. 14 and in E. B. Roche, ed., Bioreversible Carriers in Drug Design (American Pharmaceutical Association & Pergamon Press, 1987). Exemplary advantages of a prodrug can include, but are not limited to, its physical properties, such as enhanced drug stability for long-term storage.

The term “prodrug” is also meant to include any covalently bonded carriers which release the active compound in vivo when the prodrug is administered to a subject. Prodrugs of a therapeutically active compound, as described herein, can be prepared by modifying one or more functional groups present in the therapeutically active compound, including cannabinoids, such as CBGA, or a CBGA derivative, and other therapeutically active compounds used in methods according to the present invention or included in compositions according to the present invention, in such a way that the modifications are cleaved, either in routine manipulation or in vivo, to yield the parent therapeutically active compound. Prodrugs include compounds wherein a hydroxy, amino, or mercapto group is covalently bonded to any group that, when the prodrug of the active compound is administered to a subject, cleaves to form a free hydroxy, free amino, or free mercapto group, respectively. Examples of prodrugs include, but are not limited to, formate or benzoate derivatives of an alcohol or acetamide, formamide or benzamide derivatives of a therapeutically active agent possessing an amine functional group available for reaction, and the like. In some cases, the prodrug is a protecting group modified derivative of the cannabinoid compound, such as a protecting group modified CBGA or a protecting group modified derivative of CBGA.

For example, if a therapeutically active agent or a pharmaceutically acceptable form of a therapeutically active agent contains a carboxylic acid functional group, a prodrug can comprise an ester formed by the replacement of the hydrogen atom of the carboxylic acid group with a group such as C₁₋₈ alkyl, C₂₋₁₂ alkanoyloxymethyl, 1-(alkanoyloxy)ethyl having from 4 to 9 carbon atoms, 1-methyl-1-(alkanoyloxy)ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N(C₁-C₂)alkylamino(C₂-C₃)alkyl (such as (3-dimethylaminoethyl), carbamoyl-(C₁-C₂)alkyl, N,N-di (C₁-C₂)alkylcarbamoyl-(C₁-C₂)alkyl and piperidino-, pyrrolidino-, or morpholino(C₂-C₃)alkyl.

In some cases, the therapeutically active agent or a pharmaceutically acceptable form of a therapeutically active agent is a cannabinoid, such as a cannabinoid of Formula I, that contains an H at R², and the prodrug comprises a 3,6,9,12-tetraoxatridecanoyl ester; an N,N-dimethylglycyl ester; a 3,6,9,12-tetraoxatridecyl carbonate; an N-formulglycyl ester; an N-formylsarcosyl ester; a 3,6,9,12-tetraoxatridecyl oxalate; a hemisuccinate; a 4-aminobutyl carbamate; a prolyl ester; a 3-dimethylamino propionate; a glycolate; a (D)-Ribonate; a phosphate ammonium salt; an (R)-2,3-dihydroxypropyl carbonate; a 3-hydroxy-2-(hydroxymethyl)-2-methylpropanoate; a glycinate; a β-alaninate; an (S)-2,3-dihydroxypropanoate; an (S)-2,3-dihydroxypropyl carbonate; or an (R)-2,3-dihydroxypropyl carbonate at R¹.

In some cases, the therapeutically active agent or a pharmaceutically acceptable form of a therapeutically active agent is a cannabinoid, such as cannabigerolic acid A [(E)-CBGA-C5 A (cis)] of Formula I that contains a carboxylic acid functional group and the prodrug comprises a 3,6,9,12-tetraoxatridecanoyl ester; an N,N-dimethylglycyl ester; a 3,6,9,12-tetraoxatridecyl carbonate; an N-formulglycyl ester; an N-formylsarcosyl ester; a 3,6,9,12-tetraoxatridecyl oxalate; a hemisuccinate; a 4-aminobutyl carbamate; a prolyl ester; a 3-dimethylamino propionate; a glycolate; a (D)-Ribonate; a phosphate ammonium salt; an (R)-2,3-dihydroxypropyl carbonate; a 3-hydroxy-2-(hydroxymethyl)-2-methylpropanoate; a glycinate; a β-alaninate; an (S)-2,3-dihydroxypropanoate; an (S)-2,3-dihydroxypropyl carbonate; or an (R)-2,3-dihydroxypropyl carbonate derivative at R¹ of Formula I.

Similarly, if a disclosed compound or a pharmaceutically acceptable form of the compound contains an alcohol functional group, a prodrug can be formed by the replacement of the hydrogen atom of the alcohol group with a group such as (C₁-C₆)alkanoyloxymethyl, 1-((C₁-C₆))alkanoyloxy)ethyl, 1-methyl-1-((C₁-C₆)alkanoyloxy)ethyl (C₁-C₆)alkoxycarbonyloxymethyl, N(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, P(O)(O(C₁-C₆)alkyl)₂ or glycosyl (the radical resulting from the removal of a hydroxyl group of the hemiacetal form of a carbohydrate).

If a disclosed compound or a pharmaceutically acceptable form of the compound incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, C(OH)C(O)OY¹ wherein Y¹ is H, (C₁-C₆)alkyl or benzyl, C(OY²)Y³ wherein Y² is (C₁-C₄) alkyl and Y³ is (C₁-C₆)alkyl, carboxy(C₁-C₆)alkyl, amino(C₁-C₄)alkyl or mono-N or di-N,N(C₁-C₆)alkylaminoalkyl, C(Y⁴)Y⁵ wherein Y⁴ is H or methyl and Y⁵ is mono-N or di-N,N(C₁-C₆)alkylamino, morpholino, piperidin-1-yl or pyrrolidin-1-yl.

The use of prodrug systems is described in T. Jarvinen et al., “Design and Pharmaceutical Applications of Prodrugs” in Drug Discovery Handbook (S. C. Gad, ed., Wiley-Interscience, Hoboken, N J, 2005), ch. 17, pp. 733-796. Other alternatives for prodrug construction and use are known in the art. When a method or pharmaceutical composition according to the present invention, uses or includes a prodrug of cannabigerolic acid or other therapeutically active agent, prodrugs and active metabolites of a compound may be identified using routine techniques known in the art. See, e.g., Bertolini et al., J. Med. Chem., 40, 2011-2016 (1997); Shan et al., J. Pharm. Sci., 86 (7), 765-767; Bagshawe, Drug Dev. Res., 34, 220-230 (1995); Bodor, Advances in Drug Res., 13, 224-331 (1984); Bundgaard, Advanced Drug Discovery Reviews (Elsevier Press 1992); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992).

Exemplary prodrugs useful in the compositions and methods of the present invention include, but are not limited to, the following compounds (in some embodiments, prodrugs of CBGA), according to Formula II or II-A:

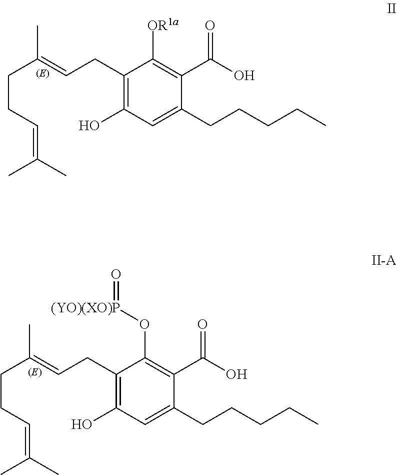

wherein R^{1a} is a prodrug moiety; and wherein X and Y can be the same or different, and are selected from the group consisting of: hydrogen, alkali metals (e.g., sodium and potassium), alkaline earth metals (e.g., calcium and magnesium); and cations of pharmaceutically acceptable organic amines (e.g., quaternated or protonated amines, including alkyl amines, hydroxyalkylamines, monoamines, diamines, and naturally occurring amines). Examples of such pharmaceutically acceptable organic bases include choline, betaine, caffeine, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, hydrabamine, isopropylamine, methylglucamine, morpholine, piperidine, polyamine resins, procaine, purines, theobromine, triethylamine, trimethylamine, tripropylamine, tromethamine, tetramethylammonium hydroxide, benzyltrimethylammonium hydroxide, tris(hydroxymethyl)aminomethane (TRIS), N-(2-hydroxyethyl)pyrrolidine, piperazine, glucosamine, arginine, lysine and histidine. In a further embodiment, X and Y are different substituent groups. In another embodiment, X and Y are the same substituent group. In an embodiment, the P(═O)(OX)(OY) group is selected from the group consisting of a diphosphate and triphosphate. In another embodiment, the compound is the salt form of the di or tri phosphate. In another embodiment, the compound is according to one of the following formulas:

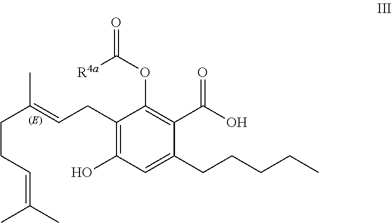

wherein R^{4a} is a straight or branched substituted or unsubstituted alkyl or R^{4a} is alkoxyalkyl, akylamine, hydroxyalkyl, or hydroxyalkylamine, preferably wherein R^{4a} comprises from 1 to 12 carbons and optionally no more than 4 substituents, more preferably wherein R^{4a} comprises from 1 to 6 carbons and optionally no more than 2 substituents;

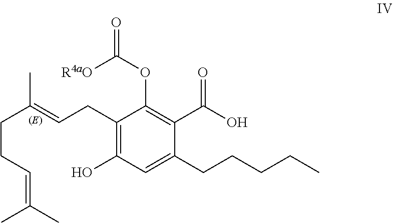

wherein R^{4a} is a straight or branched substituted or unsubstituted alkyl or R^{4a} is alkoxyalkyl, akylamine, hydroxyalkyl, or hydroxyalkylamine, preferably wherein R^{4a} comprises from 1 to 12 carbons and optionally no more than 4 substituents, more preferably wherein R^{4a} comprises from 1 to 6 carbons and optionally no more than 2 substituents;

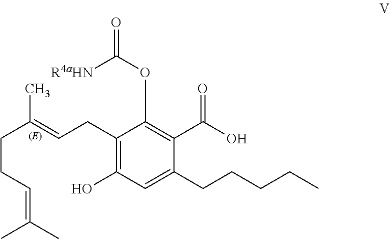

wherein R^{4a} is a straight or branched substituted or unsubstituted alkyl or R^{4a} is alkoxyalkyl, akylamine, hydroxyalkyl, or hydroxyalkylamine, preferably wherein R^{4a} comprises from 1 to 12 carbons and optionally no more than 4 substituents, more preferably wherein R^{4a} comprises from 1 to 6 carbons and optionally no more than 2 substituents;

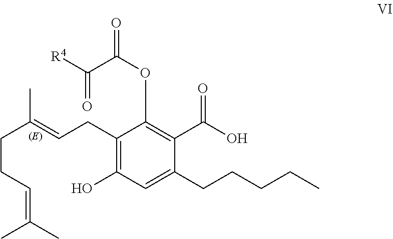

wherein R^{4a} is a straight or branched substituted or unsubstituted alkyl or R^{4a} is alkoxyalkyl, akylamine, hydroxyalkyl, or hydroxyalkylamine, preferably wherein R^{4a} comprises from 1 to 12 carbons and optionally no more than 4 substituents, more preferably wherein R^{4a} comprises from 1 to 6 carbons and optionally no more than 2 substituents.

In embodiments, prodrugs useful in the compositions and methods of the present invention include but are not limited to the following prodrug of CBGA:

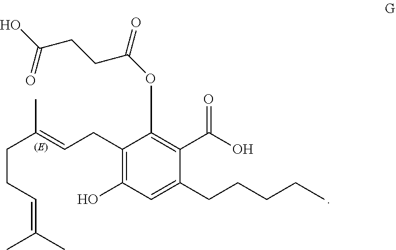 [SMILES: CCCCCc1cc(O)c(C/C=C(\C)CCC=C(C)C)c(OC(=O)CCC(=O)O)c1C(=O)O]

In embodiments, the foregoing prodrugs may be advantageously formulated with a cyclodextrin, such as random methylated beta-cyclodextrin, 2-hydroxypropyl beta-cyclodextrin, or sulfobutyl ether beta-cyclodextrin.

In embodiments, prodrugs useful in the compositions and methods of the present invention include but are not limited to the following prodrug of CBGA:

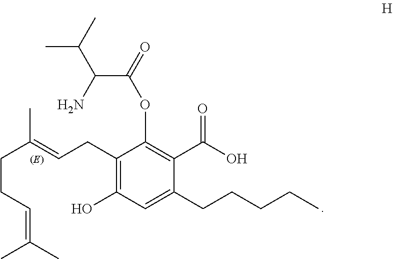 [SMILES: CCCCCc1cc(O)c(C/C=C(\C)CCC=C(C)C)c(OC(=O)C(N)C(C)C)c1C(=O)O]

In embodiments, prodrugs useful in the compositions and methods of the present invention include but are not limited to the following prodrug of CBGA:

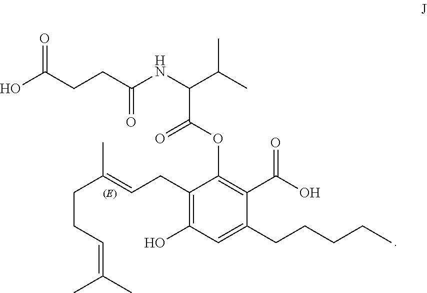 [SMILES: CCCCCc1cc(O)c(C/C=C(\C)CCC=C(C)C)c(OC(=O)C(NC(=O)CCC(=O)O)C(C)C)c1C(=O)O]

In some embodiments, prodrugs useful in the compositions and methods of the present invention include but are not limited to the following prodrug of CBGA:

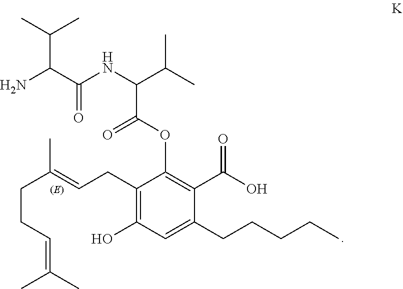 [SMILES: CCCCCc1cc(O)c(C/C=C(\C)CCC=C(C)C)c(OC(=O)C(NC(=O)C(N)C(C)C)C(C)C)c1C(=O)O]

Additional prodrug strategies for the cannabinoid compounds described herein can be found in (H. Bundgard, Design of Prodrugs (Elsevier, Amsterdam, 1988), pp. 7-9, 21-24). A discussion of prodrugs is provided in T. Higuchi et al., “Pro-Drugs as Novel Delivery Systems,” ACS Symposium Series, Vol. 14 and in E. B. Roche, ed., Bioreversible Carriers in Drug Design (American Pharmaceutical Association & Pergamon Press, 1987) the contents of which are hereby incorporated in the entirety for all purposes and in particular for the cannabinoid prodrug compositions and formulations, and methods of making, using and/or administering such prodrug compositions described therein.

Embodiment 1: In some or any embodiments, the cannabinoid compound is as disclosed in WO2020/092923 (PCT/US2019/059237), which is incorporated by reference in its entirety for all purposes. In some or any embodiments, the cannabinoid compound is accordingly to Formula VII:

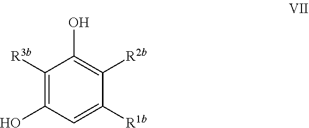

and salts thereof, wherein
R^{1b} is selected from the group consisting of C₁-C₂₀ haloalkyl, C₁-C₂₀ hydroxyalkyl, deuterated C₁-C₁₀ alkyl, tritiated C₁-C₂₀ alkyl, and C₂-C₂₀ alkenyl;
R^{2b} is selected from the group consisting of COOR^{2c} and H, preferably, COOR^{2c};
R^{2c} is selected from the group consisting of C₁-C₆ alkyl and H, preferably H; and
R^{3b} is selected from the group consisting of H and a prenyl moiety.

Embodiment 2: In an embodiment of Embodiment 1, R^{1b} is selected from the group consisting of C₅-C₁₀ haloalkyl, C₁-C₄ haloalkyl, C₁-C₁₀ hydroxyalkyl, deuterated C₁-C₁₀ alkyl, tritiated C₁-C₁₀ alkyl, and C₂-C₁₀ alkenyl. In another embodiment, R^{1b} is the group consisting of C₅-C₁₀ haloalkyl, C₁-C₄ haloalkyl, C₁-C₁₀ hydroxyalkyl, deuterated C₁-C₁₀ alkyl, and tritiated C₁-C₁₀ alkyl. In another embodiment, R^{1b} is C₅-C₁₀ haloalkyl or C₁-C₄ haloalkyl. In another embodiment, R^{1b} is selected from the group consisting of fluoropentyl, fluoroethyl, fluoropropyl, fluorobutyl, fluorohexyl, fluorooctyl, and fluorononyl. In another embodiment, R^{1b} is selected from the group consisting of 5-fluoropropyl, 4-fluorobutyl, and 3-fluoropentyl. In another embodiment, R^{1b} is C₁-C₁₀ bromoalkyl or C₁-C₁₀ chloroalkyl. In another embodiment, R^{1b} is C₁-C₁₀ hydroxyalkyl. deuterated C₁-C₁₀ alkyl or tritiated C₁-C₁₀ alkyl.

Embodiment 3: In an embodiment of Embodiment 1 or 2, R^{2b} is selected from the group consisting of COOH and H. In an embodiment of Embodiment 1 or 2, R^{2b} is COOH. In an embodiment of Embodiment 1 or 2, R^{2b} is H.

Embodiment 4: In an embodiment of any one of Embodiments 1-3, R^{3b} is H. In an embodiment of any one of Embodiments 1-3, R^{3b} is a prenyl moiety. In an embodiment, R³ is prenyl (i.e., 2-methylbut-2-en-1-yl), geranyl (i.e., 3,7-dimethylocta-2,6-diene-1-yl), farnesyl (i.e., 3,7,11-trimethyldodeca-2,6,10-triene-1-yl), or geranylgeranyl (i.e., 3,7,11,15-tetramethylhexadeca-2,6,10,14-tetraene-1-ol). In some embodiments, the prenyl moiety is geranyl. The carbon-carbon double bonds of the prenyl moiety can be in the cis (Z) configuration or trans (E) configuration. In some embodiments, R³ is trans-geranyl (i.e., (E)-3,7-di methylocta-2,6-dien-1-yl). In an embodiment of any one of Embodiments 1-3, R^{3b} is 3,7-dimethylocta-2,6-dien-1-yl (preferably in the E-configuration).

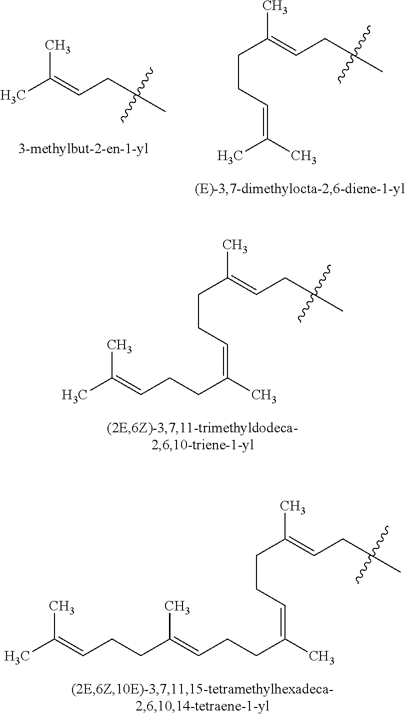

Embodiment 5: In an embodiment of Embodiment 1 or 2, the cannabinoid compound is according to Formula VIIA:

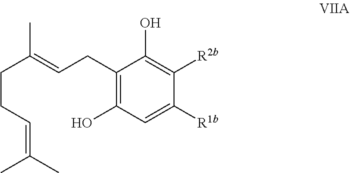

wherein R^{1b} is halopentyl, hydroxypentyl, deuterated pentyl, or tritiated pentyl and R^{2b} is COOH. In another embodiment, R^{1b} is halopentyl, hydroxypentyl, deuterated pentyl, or tritiated pentyl and R^{2b} is H.

Embodiment 6: In an embodiment of any one of Embodiments 1-5, the cannabinoid compound is not a derivative thereof.

Embodiment 7: In an embodiment of Embodiment 1, the cannabinoid compound is selected from the group consisting of:

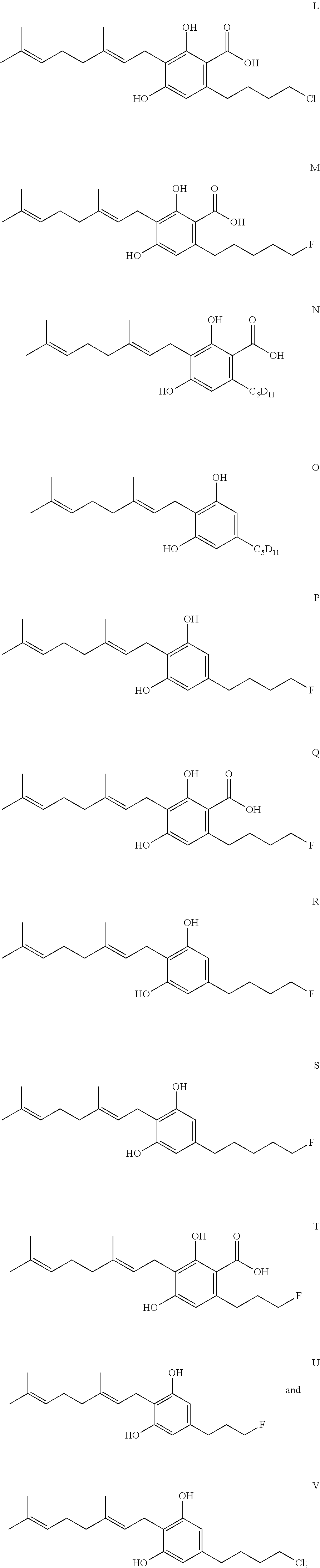

and salts thereof.

In another aspect, provided is a compound according to any one of the following formulas:

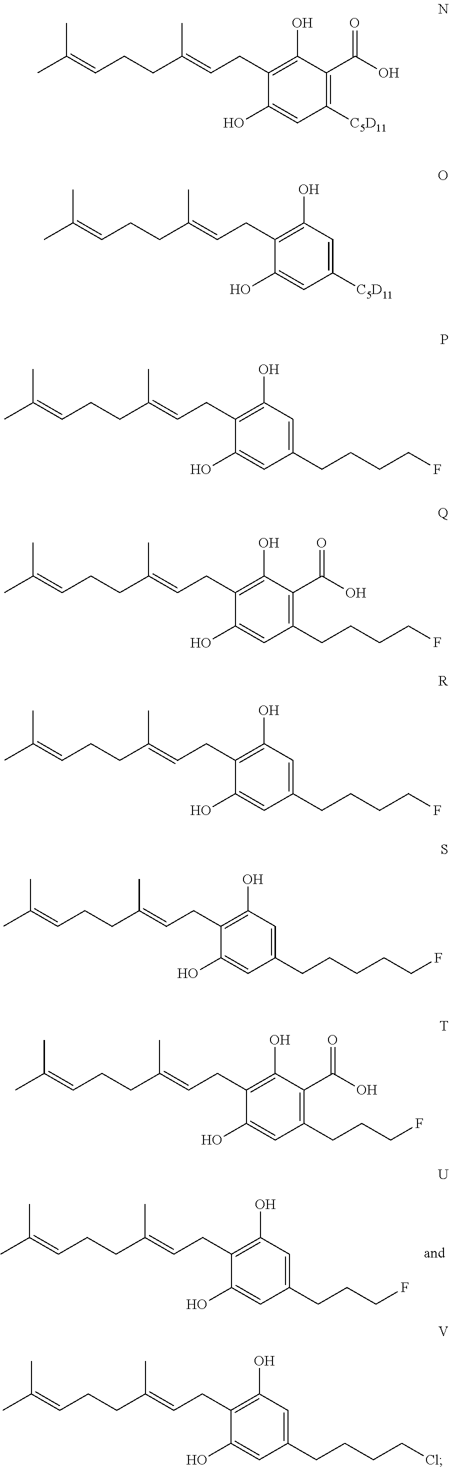

and salts thereof. In some embodiments, when the salt is present, the salt is a pharmaceutically salt thereof.

In embodiments, analogs or derivatives of these cannabinoids can be obtained by providing a precursor cannabinoid and further derivatization, e.g., by synthetic means. Synthetic cannabinoids include, but are not limited to, those described in U.S. Pat. No. 9,394,267 to Attala et al.; U.S. Pat. No. 9,376,367 to Herkenroth et al.; U.S. Pat. No. 9,284,303 to Gijsen et al.; U.S. Pat. No. 9,173,867 to Travis; U.S. Pat. No. 9,133,128 to Fulp et al.; U.S. Pat. No. 8,778,950 to Jones et al.; U.S. Pat. No. 7,700,634 to Adam-Worrall et al.; U.S. Pat. No. 7,504,522 to Davidson et al.; U.S. Pat. No. 7,294,645 to Barth et al.; U.S. Pat. No. 7,109,216 to Kruse et al.; U.S. Pat. No. 6,825,209 to Thomas et al.; and U.S. Pat. No. 6,284,788 to Mittendorf et al.

In some or any embodiments, including any of the embodiments in the foregoing paragraphs, the cannabinoid compound or the synthetic cannabinoid comprises the following structure:

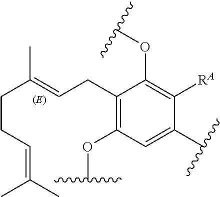

wherein R^{A} is —H, —C(O)OH, or —C(O)OR where R is C₁-C₆ alkyl.

In some cases, protecting groups can be included in compounds used in methods according to the present invention or in compositions according to the present invention. The use of such a protecting group is to prevent subsequent hydrolysis or other reactions that can occur in vivo and can degrade the compound. Groups that can be protected include alcohols, amines, carbonyls, carboxylic acids, phosphates, and terminal alkynes. Protecting groups useful for protecting alcohols include, but are not limited to, acetyl, benzoyl, benzyl, β-methoxyethoxyethyl ether, dimethoxytrityl, methoxymethyl ether, methoxytrityl, p-methoxybenzyl ether, methylthiomethyl ether, pivaloyl, tetrahydropyranyl, tetrahydrofuran, trityl, silyl ether, methyl ether, and ethoxyethyl ether. Protecting groups useful for protecting amines include carbobenzyloxy, p-methoxybenzylcarbonyl, t-butyloxycarbonyl, 9-fluorenylmethyloxycarbonyl, acetyl, benzoyl, benzyl, carbamate, p-methoxybenzyl, 3,4-dimethoxybenzyl, p-methoxyphenyl, tosyl, trichloroethyl chloroformate, and sulfonamide. Protecting groups useful for protecting carbonyls include acetals, ketals, acylals, and dithianes. Protecting groups useful for protecting carboxylic acids include methyl esters, benzyl esters, t-butyl esters, esters of 2,6-disubstituted phenols, silyl esters, orthoesters, and oxazoline. Protecting groups useful for protecting phosphate groups include 2-cyanoethyl and methyl. Protecting groups useful for protecting terminal alkynes include propargyl alcohols and silyl groups. Other protecting groups are known in the art.

### Pharmaceutical Compositions

The cannabinoid compounds described herein are typically formulated for administration. Accordingly, also provided herein is a cannabinoid compound (e.g. CBGA, a derivative thereof, or a combination thereof) formulated for administration with one or more pharmaceutically acceptable carrier(s), diluent(s), or excipient(s). The pharmaceutical compositions may be prepared by known procedures using well-known and readily available ingredients.

Pharmaceutical compositions comprising the cannabinoid compounds of the subject invention may be formulated for administration to a subject by one of a variety of standard routes, for example, intra-cerebroventricularly, intrathecally, intra-nasally, orally, topically, parenterally, by inhalation or spray, in dosage unit formulations containing conventional non-toxic pharmaceutically acceptable carriers, adjuvants, excipients and/or vehicles.

The term parenteral as used herein includes in various embodiments subcutaneous injections, intradermal, intra-articular, intravenous, intramuscular, intravascular, intrasternal, intrathecal injection and infusion techniques. The pharmaceutical composition will typically be formulated in a format suitable for administration to the subject by the selected route, for example, as an injection, elixir, tablet, troche, lozenge, hard or soft capsule, pill, orally disintegrating film, intranasal spray, suppository, oily or aqueous suspension, dispersible powder or granule, emulsion, injectable, or solution.

In certain embodiments, the pharmaceutical compositions are formulated for administration via a systemic route, for example, intravenously, intramuscularly, intradermally, intraperitoneally, subcutaneously, or orally.

Pharmaceutical compositions for intranasal administration may also be presented as aerosol. Pharmaceutical compositions for oromucosal spray use may also be presented as either buccal, sublingual, or oropharyngeal administration. Pharmaceutical compositions for sublingual use may also be presented as liquid tincture, lozenges, pastilles, tablets, troche or as orally disintegrating film applied under the tongue. Oral, mucosal, oromucosal sprays, intranasal, pulmonary, topical and transdermal and other routes of administration for cannabinoids have been described (see for example, Bruni et al., Molecules, 23: 2478; doi:10.3390/molecules23102478 and WO2007032962A2).

Pharmaceutical compositions intended for oral use may be prepared in either solid or fluid unit dosage forms. Fluid unit dosage form can be prepared according to procedures known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. An elixir is prepared by using a hydroalcoholic (for example, ethanol) vehicle with suitable sweeteners such as sugar or saccharin, together with an aromatic flavoring agent. Suspensions can be prepared with an aqueous vehicle with the aid of a suspending agent such as acacia, tragacanth, methylcellulose and the like.

Solid formulations such as tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate: granulating and disintegrating agents for example, corn starch, or alginic acid: binding agents, for example starch, gelatin or acacia, and lubricating agents, for example magnesium stearate, stearic acid or tale and other conventional ingredients such as dicalcium phosphate, magnesium aluminum silicate, calcium sulfate, starch, lactose, methylcellulose, and functionally similar materials. The tablets may be uncoated or they may be coated by known techniques to delay disintegration and absorption in the gastrointestinal tract and thereby provide a sustained action over an extended period of time. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Soft gelatin capsules are prepared by machine encapsulation of a slurry of the compound with an acceptable vegetable oil, light liquid petrolatum or other inert oil.

Aqueous suspensions contain the active ingredient in admixture with one or more excipients suitable for the manufacture of aqueous suspensions. Such excipients include suspending agents, for example sodium carboxylmethylcellulose, methyl cellulose, hydropropylmethylcellulose, sodium alginate, polyvinylpyrrolidone, gum tragacanth and gum acacia; and dispersing or wetting agents such as naturally-occurring phosphatides (for example, lecithin), condensation products of an alkylene oxide with fatty acids (for example polyoxyethylene stearate), condensation products of ethylene oxide with long chain aliphatic alcohols (for example hepta-decaethyleneoxycetanol), condensation products of ethylene oxide with partial esters derived from fatty acids and a hexitol (for example, polyoxyethylene sorbitol monooleate), or condensation products of ethylene oxide with partial esters derived from fatty acids and hexitol anhydrides (for example polyethylene sorbitan monooleate). The aqueous suspensions may also contain one or more preservatives, for example ethyl, or n-propyl-p-hydroxy benzoate, one or more coloring agents, one or more flavoring agents or one or more sweetening agents, such as sucrose or saccharin.

Oily suspensions may be formulated by suspending the active ingredient in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oil phase may be a vegetable oil, for example olive oil or peanut oil, or a mineral oil, for example liquid paraffin, or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of such partial esters with ethylene oxide, for example polyoxyethylene sorbitan monooleate. The emulsions may also optionally contain sweetening and flavoring agents.

The pharmaceutical compositions may be in the form of a sterile injectable aqueous or oleaginous suspension. Such suspensions may be formulated as known in the art using suitable dispersing or wetting agents and suspending agents such as those mentioned above. The sterile injectable preparation may also be a sterile injectable solution or a suspension in a non-toxic parentally acceptable diluent or solvent, for example as a solution in 1,3-butanediol. Other acceptable vehicles and solvents that may be employed include, for example, water, Ringer's solution and isotonic sodium chloride solution. In addition, sterile, fixed oils may be employed as a solvent or suspending medium. Various bland fixed oils known to be suitable for this purpose may be employed including synthetic mono- or diglycerides. In addition, fatty acids such as oleic acid find use in the preparation of injectables. Adjuvants such as local anesthetics, preservatives and buffering agents may also optionally be included in the injectable solution or suspension.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, Pa. (2003).

The concentration of the cannabinoid compound (e.g., cannabigerolic acid) in the formulation will vary depending on the condition to be treated and/or the mode of administration.

### Methods of Use

Described herein are methods of protecting a neuron from neurodegeneration, as well as methods for stimulating neuritogenesis, e.g. by promoting neurite elongation and/or restoring neurite formation. In general, the methods include contacting an affected population of neurons with a therapeutically effective amount of CBGA, a CBGA derivative, prodrugs thereof, or combinations thereof. The method can be an in vitro method. Alternatively, the method can be a method performed at least partially in vivo, such as by administering a neuroprotective composition to a subject. The administering can be performed by intranasal, sublingual, systemic (intravenous injection) or localized (intracerebroventricular or subcutaneous injections). The administering can be performed by a localized administration method that is non-invasive. For example, localized administration may be directly to brain.

In certain embodiments, the compound is administered for a period of less than six weeks. In certain embodiments, the compound is administered for a period of about one to four weeks. In other embodiments, such as to treat a neurodegenerative disease such as AD, PD, ALS, HD, MS the compound will be administered for an extended period of time, such as for several years, or for the remaining life of the patient. The compound may be administered weekly, every other day, daily, twice per day, or three times per day.

The cannabinoid compound may be administered to treat the brain of a subject in need of treatment to protect brain neurons. For example, the subject may have received an “insult” affecting the brain nerves, such as a physical injury. As another example, the subject may have received a diagnosis of AD (pre-clinical stage) or may be suffering from the mild to severe clinical symptoms of AD. If the cannabinoid compound is administered to protect neurons, such as brain neurons, then the cannabinoid compound can be administered at a dosage that provides a peak (e.g. Cₘₐₓ), median (e.g. steady state), or trough (e.g. C_{trough}), preferably peak, neuroprotective effective concentration of the cannabinoid compound (e.g., CBGA or a derivative thereof) in contact with the target neuron or target neuronal population. In some embodiments, the target neuron is a brain neuron. In some embodiments, the target neuron is a peripheral neuron. In some embodiments, the target neuron is a central neuron.

In an embodiment, the neuroprotective effective concentration of the cannabinoid compound (e.g., CBGA or a derivative or a prodrug thereof) in contact with the target neuron or target neuronal population is less than about 20 μM, less than about 15 μM, less than about 14 μM, less than about 13 μM, less than about 12 μM, less than about 10 μM, less than about 5 μM, less than about 1.5 μM, less than about 0.5 μM or less than about 0.15 μM. In an embodiment, the neuroprotective effective concentration of the cannabinoid compound (e.g., CBGA or a derivative thereof) in contact with the target neuron or target neuronal population is from greater than about 0.15 μM to less than about 20 μM, or from greater than 0.15 μM to less than 20 μM, or from at least about 0.15 μM to less than about 20 μM, or from at least 0.15 μM to less than 20 μM, or from greater than about 0.15 μM to less than about 15 μM, or from greater than 0.15 μM to less than 15 μM, or from at least about 0.15 μM to less than about 15 μM, or from at least 0.15 μM to less than 15 μM, 0.15 μM to less than about 10 μM, or from greater than 0.15 μM to less than 10 μM, or from at least about 0.15 μM to less than about 10 μM, or from at least 0.15 μM to less than 10 μM, 0.15 μM to less than about 5 μM, or from greater than 0.15 μM to less than 5 μM, or from at least about 0.15 μM to less than about 5 μM, or from at least 0.15 μM to less than 5 μM.

In an embodiment, the amount of a cannabinoid compound (e.g., CBGA or a derivative thereof) sufficient to inhibit or slow the progression of neurodegenerative disease is an amount that results in a concentration of about 0.15 μM to about 15 μM, from about 0.15 μM to about 10 μM, from about 0.15 μM to about 7.5 μM, or from about 0.15 μM to about 5 μM in contact with the neuron. In other embodiments, the amount of a cannabinoid compound (e.g., CBGA or a derivative thereof) sufficient to inhibit or slow the progression of neurodegenerative disease is an amount that results in a concentration of about 0.5 μM to about 15 μM, from about 0.5 μM to about 10 μM, from about 0.5 μM to about 7.5 μM, or from about 0.5 μM to about 5 μM in contact with the neuron.

In an embodiment, for i.c.v. administration in a neurodegenerative disease indication (e.g., to treat AD), the i.c.v. dose can be from about 11 μg to about 1.4 mg, from about 11 μg to about 1.0 mg, from about 11 μg to about 0.5 mg, from about 11 μg to about 0.25 mg, from about 11 μg to about 0.125 mg, from about 5.5 μg to about 1.0 mg, from about 5.5 μg to about 0.5 mg, from about 5.5 μg to about 0.25 mg or from about 5.5 μg to about 0.125 mg applied to the brain, such as in the form of an injectable formulation. The dose can be repeated, e.g., weekly, every other day, daily, or twice a day. In an embodiment, for i.c.v. administration in a neurodegenerative disease indication (e.g., to AD), the brain dose can be from 1.1 μg to about 0.14 mg, from 1.1 μg to about 0.10 mg, from 1.1 μg to about 0.05 mg from 1.1 μg to about 0.025 mg or from 1.1 μg to about 0.0125 mg applied to the brain, such as in the form of an i.c.v. injection or via pump. The dose can be repeated, e.g., weekly, every other day, daily, or twice a day.

In certain embodiments, the cannabinoid compound, or a formulation thereof, is administered to a subject having AD. In certain embodiments, the cannabinoid compound, or a formulation thereof, is administered to a subject having ALS, HD, PD, or MS. In certain embodiments, the cannabinoid compound, or a formulation thereof, is administered to a subject having mild to severe symptoms of AD.

The cannabinoid compound may be administered to treat a subject in need of treatment to protect peripheral neurons. For example, the subject may have received an insult affecting one or more peripheral nerves, such as a physical injury. As another example, the subject may have a disease or condition characterized by peripheral nerve degeneration. PNS disorders outside the brain and spinal cord that would benefit from the subject invention also include entrapment neuropathy, such as carpal tunnel syndrome; brachial plexus injury, such as that seen in a motorcycle upper extremity traction injury; and direct open traumatic injury. Peripheral nerve disorders distort or interrupt the messaging between the brain and the rest of the body and can affect one nerve or many nerves. Some are the result of other diseases, like diabetic nerve problems. Others, like Guillain-Barre syndrome, happen after a virus infection. Still others are from nerve compression, like carpal tunnel syndrome or thoracic outlet syndrome. In some cases, like complex regional pain syndrome and brachial plexus injuries, the problem begins after an injury. Some peripheral nerve disorders are hereditary. A group of hereditary disorders, such as hereditary sensory and autonomic neuropathies (HSANs) are caused by PNS dysfunction. One such disorder, familial dysautonomia, is caused by mutation of the IKBKAP gene.

The cannabinoid compound may be administered to treat a subject in need of treatment to protect central neurons. For example, the subject may have received an insult affecting neurons in the central nervous system (CNS). As another example, the subject may have a disease or condition characterized by central nerve degeneration.

The method may include or further include administering a second active agent simultaneously or sequentially in combination with the cannabinoid compound provided herein. In some cases, the second active agent is a therapeutic agent for the treatment of Alzheimer's Disease.

The pharmaceutical composition can contain additional active agents. In some embodiments, the pharmaceutical composition can contain CBGA, or a derivative thereof, and an additional cannabinoid or a terpenoid. In some embodiments, the pharmaceutical composition can contain an additional active pharmaceutical agent for treatment of AD, PD, ALS, HD and MS or an additional active pharmaceutical agent for treatment of neurodegenerative disease.

Currently, different classes of therapeutic agents are used for treatment and reversal of AD, including but not limited to: FDA-approved medications available for AD that are designed to relieve symptoms such as memory loss for a limited time. These drugs, including Aricept, Exelon, Razadyne, Galantamine, Donepezil, Tacrine and Rivastigmine are acetylcholinesterase inhibitors that increase levels of acetylcholine, a neurotransmitter that sends signals from one brain cell to another.

Namenda works by regulating the activity of neurotransmitter glutamate. Namzaric combines the two approaches. None of these drugs can stop damage to brain cells; they may alleviate memory issues for a short time by regulating neurotransmitters. There are currently >120 potential drugs in clinical trials designed to treat the underlying causes of Alzheimer's, rather than its symptoms. J147, an experimental anti-aging mitochondrial ATP synthase modulator, is the most promising. Nonetheless, any therapeutic agent appropriate for treating AD may be used in concert with the compound of any one of Formulas I-VIIA and A-F. Nonetheless, any therapeutic agent appropriate for treating AD may be used in concert with the compound of Formula I and derivatives thereof.

In some embodiments, the pharmaceutical compositions described herein, e.g., containing CBGA or a derivative thereof, allow a lower dose, or less frequent dosing, of one or more therapeutic agents for the treatment of AD.

The cannabinoid compounds may be also administered to stimulate neuritogenesis in a patient in need thereof. Neuritogenesis is crucial in ensuring proper synaptogenesis, axon guidance, and neuronal function, and can play a role in ischemic stroke (Arvidsson et al., 2002; Zhanget al., 2004) as well as spinal cord injuries that result in axonal injury or degeneration of neurites. Improper neuritogenesis also underlies a variety of neurodevelopmental disorders such as (Cellular and Molecular Life Sciences (2020) 77:1511-1530) schizophrenia, Down syndrome, and autism spectrum disorder (ASD). Degeneration and loss of spinal motor neurons can also cause progressive and fatal motor neuron diseases such as amyotrophic lateral sclerosis (ALS), primary lateral sclerosis, and spinal muscular atrophy. Also contemplated herein is the use of the subject compounds, compositions and methods in regenerative therapies for motor neuron diseases (Cells 2020, 9(4), 934; https://doi.org/10.3390/cells9040934), as well as chronic hearing loss, tinnitus, hyperacusis, presbycusis, or balance disorders associated with cochlear synaptopathy and vestibular synaptopathy.

### Examples

### Example 1: Protection of Differentiated SH-SY5Y Neuronal Cells with Cannabigerolic Acid

Cell Culture and Differentiation: The SH-SY5Y human neuronal cell line was derived from neuroblastoma established from a metastatic bone tumor (patient with neuroblastoma). The cells were maintained in DMEM:F12 culture medium supplemented with 10% FBS and 1% Antibiotic-Antimycotic penicillin/streptomycin (growth medium) at 37° C. in a humidified atmosphere of 5% CO₂. To induce neuronal differentiation of SH-SY5Y cells, culture media was replaced by a growth medium containing Retinoic Acid (RA) for 4-5 days at 37° C. in a humidified atmosphere of 5% CO₂.

Compounds and Dosing Formulations: Selected cannabinoids: CBD, CBDA, CBC, CBG, CBGA, CBN, CBND and Δ⁹-THC were procured from Cayman Chemicals and TRC. Ethanol (100%) was used as the solvent to prepare 1 and 10 mM stock solutions. Treatment concentrations for CBGA were prepared at 0.5 μM, 1.5 μM, 5 μM, 10 μM, 15 μM, 20 μM. Treatment concentrations for CBD, CBDA, CBC, CBN, CBND and Δ⁹-THC were prepared at 0.5, 1.5, 5 and for CBG at 0.5 μM, 1.5 μM, 5 μM, 10 μM, 15 μM. Treatments were prepared directly in the control medium (DMEM:F12+5% FBS+1% Antibiotic-Antimycotic) by using appropriate stock solutions.

Evaluation of Cytotoxicity and Neuroprotection: Evaluation of cannabinoids neuroprotection and cytotoxicity on differentiated SH-SY5Y human neuronal cells was carried out by MTT (3-(4,5-dimethylthiazol-2-yl)-2,5-diphenyltetrazolium bromide) assay. Initially, cannabinoid cytotoxicity and neuroprotection were evaluated in a primary screen assay without the presence of cytotoxicity inducing insult agents. Subsequently, cannabinoids with “border-line” cytotoxicity were selected and advanced to a secondary screen by further evaluation in the presence of insult-inducing cytotoxic agents such as Staurosporin (STSR) and Amyloid-beta (Aβ1-42) peptide. The Retinoic Acid (RA) differentiated SH-SY5Y neuronal cells were seeded onto 96-well plates (10,000 cells/well) in DMEM:F12 complete medium. Cells were differentiated for 4-5 days in the presence of RA. Post-differentiation cells were treated with STSR (72 hrs) or Aβ1-42 (24 hrs) and processed for MTT assay.

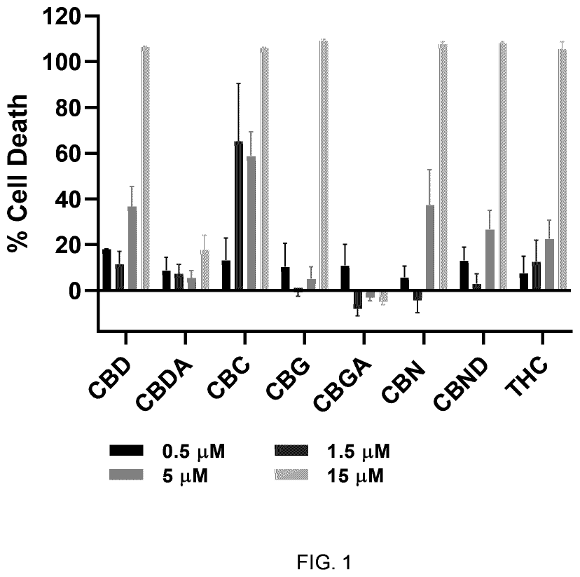

MTT Assay: For the MTT assay, differentiated SH-SY5Y neuronal cells were treated with cannabinoids at various concentrations for 72 hrs in the presence of STSR at 75 nM, 100 nM, 150 nM and 200 nM or for 24 hrs in the presence of A 1-42 peptide at 5 μM, respectively and processed to determine cytotoxicity. Briefly, 5 mg/mL of methylthiazolyldiphenyl-tetrazolium bromide (Sigma-Aldrich) stock solution was prepared in PBS. Following treatment of SH-SY5Y neuronal cells with cannabinoids for 72 hrs, the cells were incubated with 20 μL of MTT stock solution in 200 μL DMEM for 2 hrs at 37° C. Following subsequent washes with PBS, 200 μL of isopropanol was added to each well. The resulting change in color from dissolving formazan salt was immediately quantified using a spectrophotometer (BMG Labtech) at a wavelength of 570 nm. The data were normalized to either Vehicle Controls (VC) containing DMSO/Ethanol for STSR and NH₄OH/Ethanol for Aβ1-42 peptide or it was normalized to STSR or A 1-42 peptide alone and presented as % Cell Death. The results are illustrated in FIGS. 1-7.

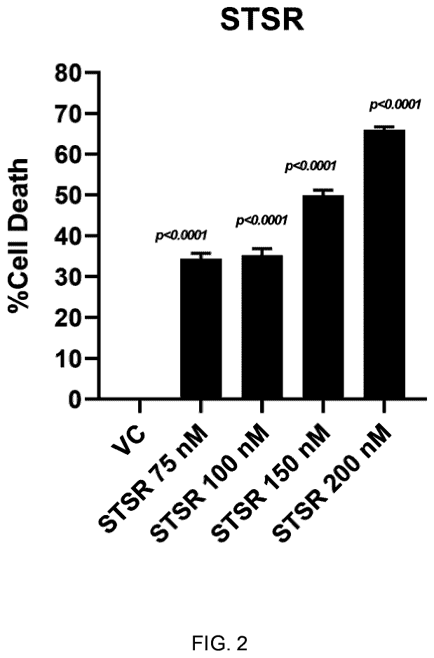

As shown in FIG. 1, following a primary screen of eight different cannabinoids for cytotoxicity at 0.5 μM, 1.5 μM, 5 μM and 15 μM concentrations CBGA was identified as the only cannabinoid exhibiting pro-survival neuroprotective effect at 1.5 μM, 5 μM and 15 μM when in contact with differentiated SH-SY5Y cells. In contrast, other cannabinoids such as CBD, CBDA, CBC, CBG, CBN, CBND and THC exhibited high levels of toxicity when in contact with differentiated SH-SY5Y cells at a concentration range of about 0.5 μM to 1.5 μM (CBD, CBDA, CBC and THC) or at 5 M to 15 μM (CBD, CBDA, CBC, CBG, CBN, CBND and THC). As shown in FIG. 2, treatment of differentiated SH-SY5Y cells—with cytotoxicity-inducing insult agent STSR at 75 nM, 100 nM, 150 nM and 200 nM resulted in a highly significant dose-dependent cytotoxicity (p<0.0001) at all concentrations. The minimum cytotoxicity effect was observed at 75 nM (˜34% cell death) and a maximum at 200 nM (˜66% cell death). The Vehicle Control (VC) cytotoxicity (equivalent to DMSO/Ethanol) for 200 nM and 150 nM concentrations was at ˜6-9% cell death, respectively.

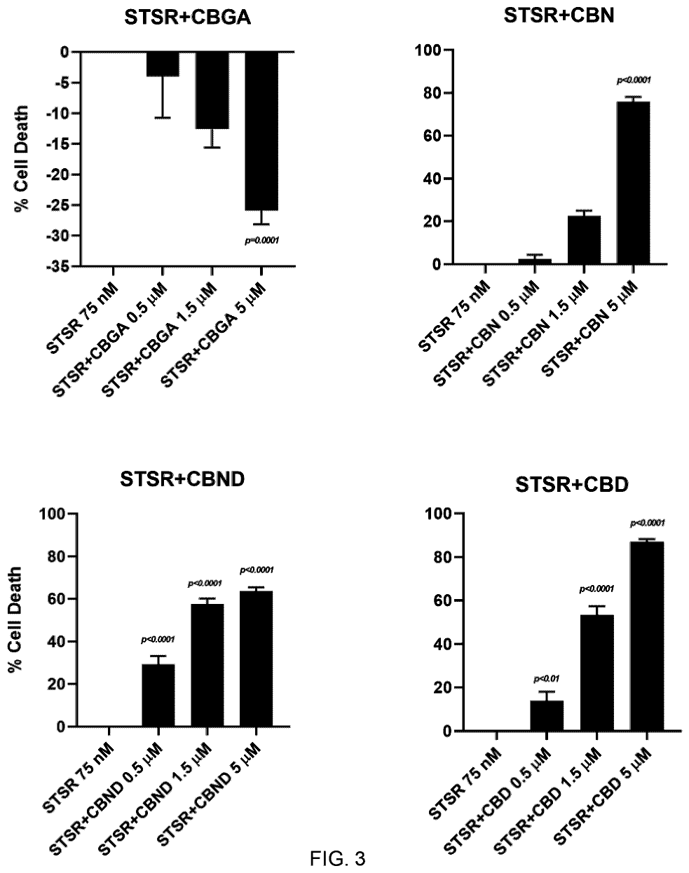

CBGA exhibited significant neuroprotection against STSR-induced cytotoxicity when in contact with differentiated SH-SY5Y cells at 0.5 μM, 1.5 μM and 5 μM concentrations. The neuroprotective effect of CBGA was statistically significant at 5 μM (p=0001). In contrast, other cannabinoids, such as CBN, CBND and CBD, used at the same concentration range did not protect differentiated SH-SY5Y cells from STSR-induced insult. They exhibited a high level of toxicity when in contact with neuronal cells (FIG. 3). Such neuroprotective effect CBGA from cytotoxicity under these conditions could potentially prevent neuronal damage in the setting of neurodegenerative diseases.

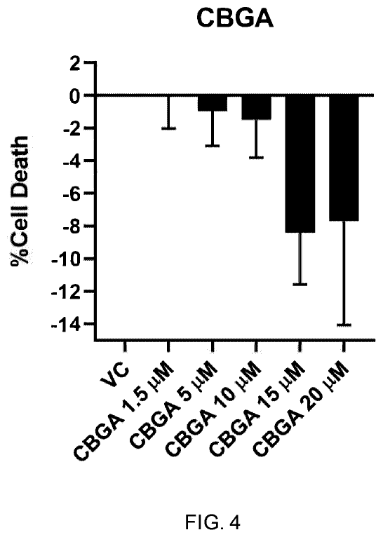

Treatment of differentiated SH-SY5Y cells with CBGA alone at high concentrations up to 20 μM was safe, did not result in cell death and moreover, induced dose-dependent cell proliferation (FIG. 4).

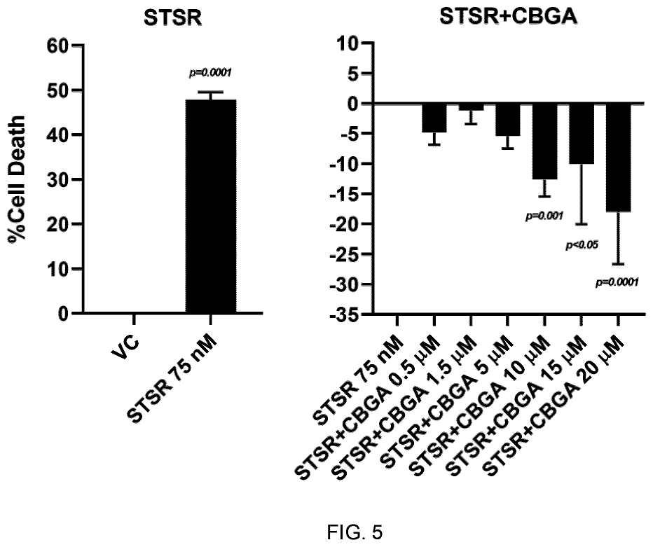

The neuroprotective effect of CBGA against STSR-insult was also observed at a much broader concentration range. As shown in FIG. 5, when in contact with differentiated SH-SY5Y cells, STSR-insult alone at 75 nM induced approximately ˜48% cell death. Concurrent exposure with CBGA from 0.5 μM to 20 μM resulted in a significant dose-dependent neuroprotection against insult-induced cytotoxicity and was statistically significant at 10 μM (p=0.001), 15 μM (p<0.05) and 20 μM (p=0.0001) concentrations.

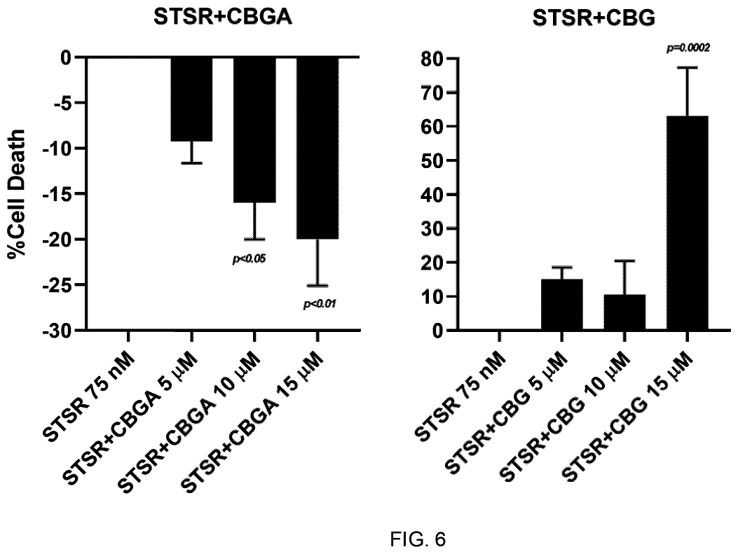

The neuroprotective effect of CBGA was compared to that of CBG, its de-carboxylated cannabinoid derivative. When used at the same concentration range of 5 μM, 10 μM and 15 μM, CBGA protected differentiated SH-SY5Y cells from STSR induced cytotoxicity at 75 nM. However, CBG did not protect the differentiated SH-SY5Y cells at 5 μM, 10 μM and 15 μM against STSR induced cytotoxicity at 75 nM (FIG. 6).

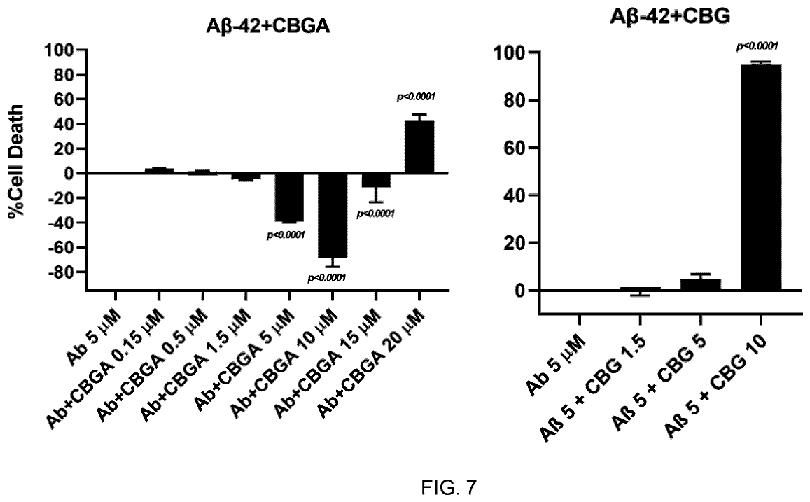

CBGA also conferred effective neuroprotection against Aβ1-42 induced cytotoxicity insult on SH-SY5Y neuronal cells. FIG. 7 illustrates comparison of the neuroprotective effects of CBGA versus CBG on differentiated SH-SY5Y cells from the Aβ1-42 induced cytotoxicity at 5 μM. Aβ1-42 insult induced approximately ˜23-52% cytotoxicity of SH-SY5Y cells. Concurrent exposure with CBGA at 0.15 μM, 0.5 μM, 1.5 μM, 5 μM, 10 μM and 15 μM concentrations protected cells from Aβ1-42 induced insult in a dose-dependent manner. This neuroprotection was highly significant at 5 μM, 10 μM and 15 μM concentrations, respectively (p<0.0001). Treatment of cells at the highest Aβ+CBGA 20 μM concentration resulted in cell death. Concurrent exposure with CBG at 1.5 μM, 5 μM and 10 μM concentrations did not protect differentiated SH-SY5Y cells from Aβ1-42 induced insult and resulted in significant cytotoxicity at 10 μM (p<0.0001). Overall, this data indicates that the beneficial therapeutic range for CBGA neuroprotection against STSR insult is between 0.5 μM and 20 μM and against Aβ1-42 insult is at greater than 0.15 μM and less than 20 μM.

### Example 2: Receptor Antagonist Study

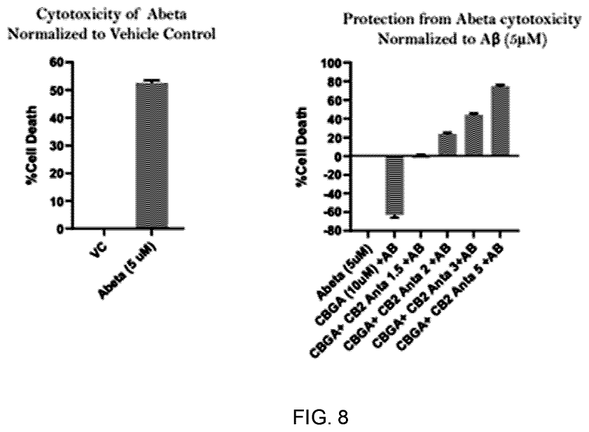

FIG. 8 shows that the CB2 antagonist SRI 14528 has a predominant effect on the reversal of CBGA mediated protection. At the lowest concentration tested (1.5 μM), the CB2 antagonist completely reversed the CBGA mediated neuroprotection of the SH-SY5Y cells.

### Example 3: Role of CBGA in Regulating Apoptosis Pathways

For Western blot analysis, control and the treated cells were harvested and lysed in cold lysis buffer (150 mM NaCl, 50 mM Tris-HCl, 1% Triton X-100, 0.1% SDS, 0.5% Sodium deoxycholate, and 1% protease-phosphatase inhibitor mixture). The supernatants were collected and protein concentrations were determined using Bio-Rad protein assay (Bio-Rad Laboratories). Whole cell lysates (10 μg) were subjected to 10% SDS-polyacrylamide electrophoresis and transferred to nitrocellulose membrane. The membranes were blocked with 5% skim milk in TBS-T (Tris-buffered saline with 0.05% Tween-20) for 1 h at RT and blotted overnight in the presence of monoclonal anti-mouse MAP2 (1:1000, Sigma, cat. no. M1406), Tau (1:1000, Invitrogen, cat. no. AHB0042), TUJI (1:5000, BioLegend, cat. no. MMS-435P), BAX (1:1000, Cell signaling, cat. no. 2772) and rabbit polyclonal total and phosphorylated BCL (1:1000, Cell signaling, antibodies #2827 and 2870). The membranes were washed and incubated for 1 h in RT with either horseradish peroxidase (HRP)-conjugated goat anti-mouse (1:2000) or goat anti-rabbit antibodies (1:2000) (Jackson Lab). The membranes were washed and developed using chemiluminescence detection kit (Millipore, Billerica, MA, USA) on Alpha Innotech FluorChem 8800. β-Actin or Vinculin was used as a loading control. Densitometric analysis of protein expression levels was performed using ImageJ Software.

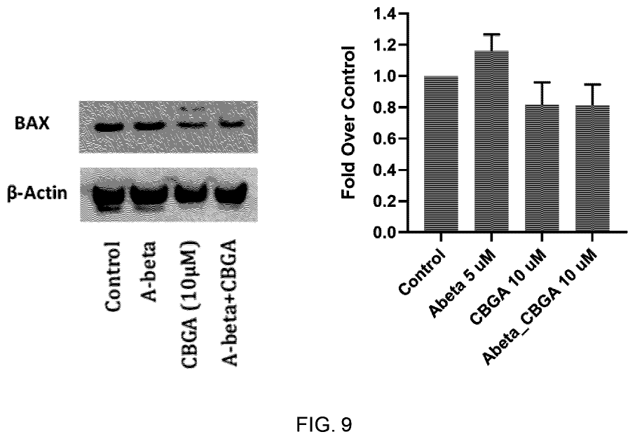

CBGA mediated effects on apoptotic markers in Aβ1-42 induced cytotoxicity in SH-SY5Y cells. Aβ1-42 (5 μM) treatment of differentiated SH-SY5Y cells induced upregulation of BCL2-like protein 4 (BAX) a pro-apoptotic marker. BAX is known in the art (Oltvai et al., (1993) Cell. 74 (4): 609-619). As shown in FIG. 9, CBGA treatment alone or in the presence of Aβ1-42 attenuates BAX expression to the control levels. This observation supports an anti-apoptotic role of CBGA.

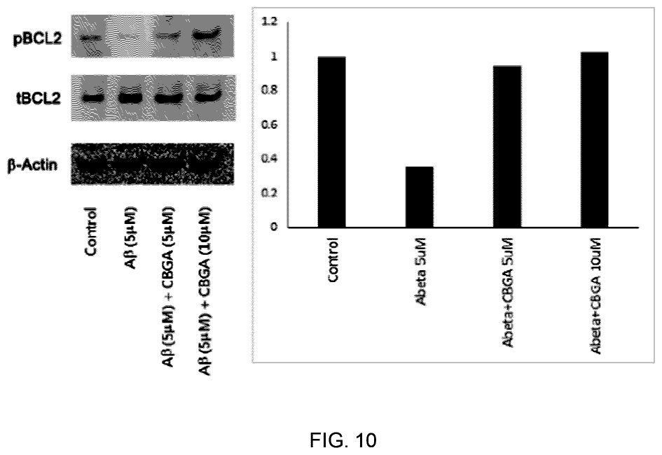

Differentiated SH-SY5Y cells that were subjected to Aβ1-42 “insult” showed downregulation of B-cell lymphoma 2 (BCL2, an anti-apoptotic marker). As shown in FIG. 10, application of CBGA at both 5 μM and 10 μM to Aβ1-42 (5 μM) treated SH-SY5Y cells abrogates the downregulation of BCL2 induced by Aβ1-42 alone. Restoration of BCL2 back to the control levels with the application of CBGA illustrates the anti-apoptotic role of CBGA.

### Example 4: Evaluation of Changes in CB1 and CB2 Receptor Expression in the Presence of CBGA

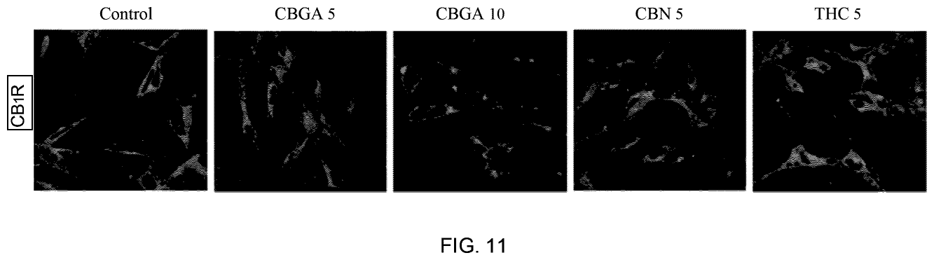

FIG. 11 shows that CBGA promotes the internalization and translocation of CB1R in Golgi apparatus in a dose-dependent manner.

### Example 5: Changes in TuJ1 Expression in the Presence of CBGA

Tuj1 Tubulins are building blocks of microtubules. As such, TuJ1 expression can reveal the fine details of axonal structures and dendrites. Therefore, changes in TuJ1 expression can be directly correlated with neuronal health and communication.

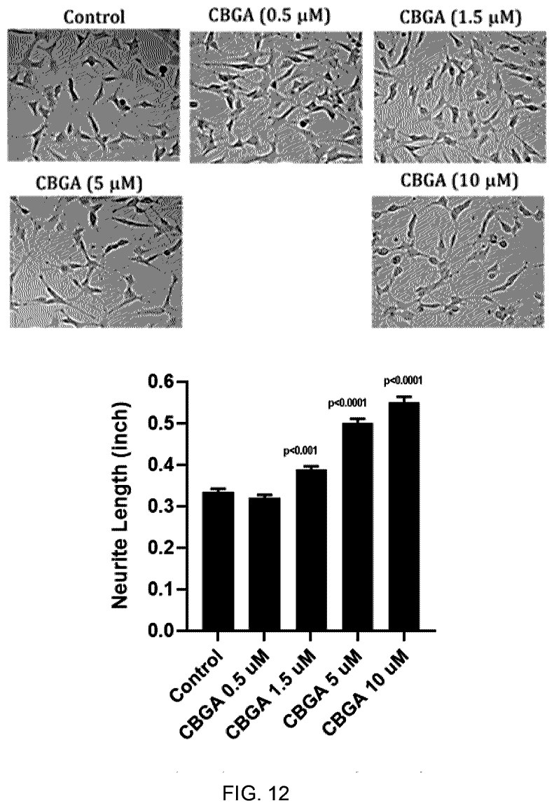

Changes in neurite length following treatment with CBGA in a dose dependent manner was determined. Note that CBGA in a dose dependent manner significantly increased the neuritogenesis in SH-SY5Y cells (FIG. 12).

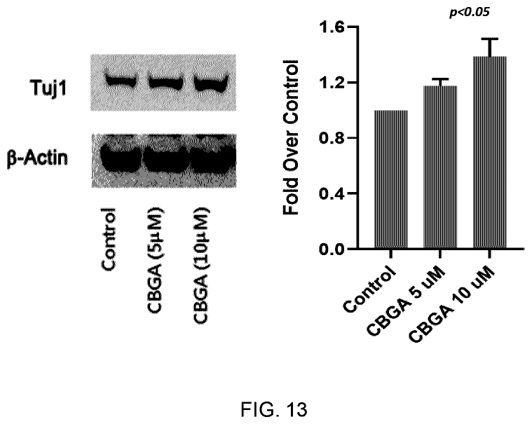

Changes in TuJ1 expression were followed in differentiated SH-SY5Y cells exposed to CBGA. FIG. 13 shows a Western blot analysis depicting the changes in TuJ1 expression in response to CBGA. CBGA at 5 μM and 10 μM promotes upregulation of TuJ1 expression.

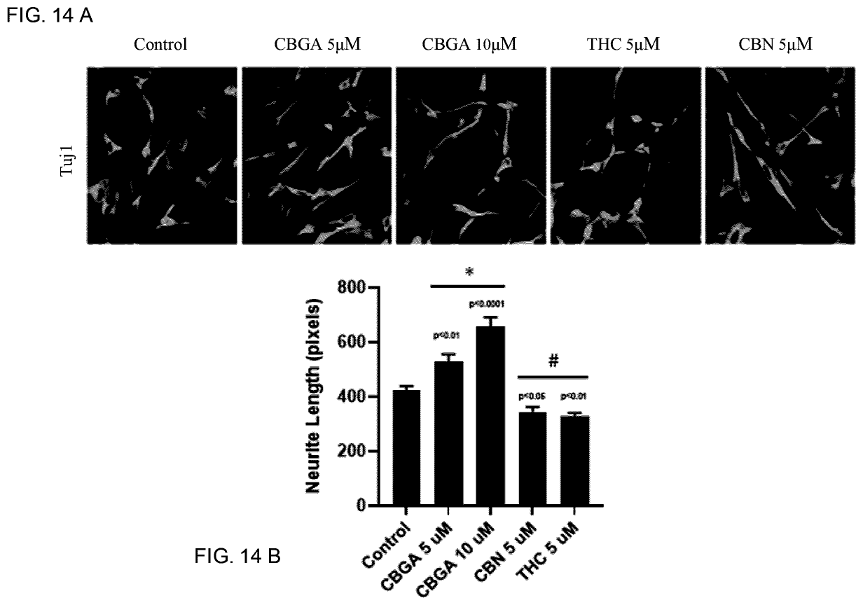

FIG. 14 shows representative immunocytochemical photomicrographs depicting the changes in TuJ1 expression in response to CBGA. CBGA at 5 μM and 10 μM promotes upregulation of TuJ1 expression.

Other cannabinoids do not show the same positive effects on neuronal growth and TuJ1 expression. Two other cannabinoids, THC and CBN, were tested. FIG. 14 shows the results of the experiments. FIG. 14 provides representative immunocytochemical photomicrographs showing the effects of changes in TuJ1 expression. THC (5 μM) treatment disrupted neuronal morphology and neurite outgrowth as indicated by punctured TuJ1 staining in the neurites. A significant loss in neurite formation and arborization was also observed. CBN (5 μM) treatment resulted in the loss of neurite outgrowth and elongation as well as significantly shorter neurites and arborization compared to either control or CBGA (5 μM and 10 μM) treated cells.

### Example 6: CBGA Mediated Effects on MAP2 Neuritogenesis Markers in SH-SY5Y Cells Subject to Aβ1-42 Induced Cytotoxicity

Microtubules are polar rod-like cytoskeletal elements that are involved in neurite development. By crosslinking with intermediate filaments and other microtubules, MAP2 stabilizes microtubule growth.

MAPs occur at high levels in neurons where their expression is under strong developmental regulation, suggesting that they are involved in neuronal morphogenesis (see e.g., Matus (1991) J. Cell Sci. Suppl. 15:61-67). In particular, MAP2 isoforms are involved in microtubule assembly, which is an essential step in neuritogenesis. Thus, MAP-2 plays a role in neurite outgrowth during neuronal development and stabilizes dendritic shape.

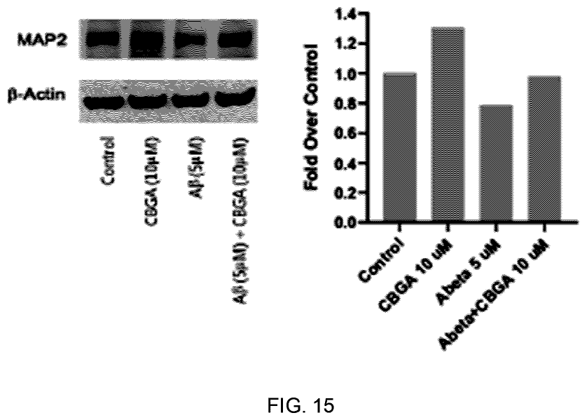

Representative western blot in FIG. 15 depict the changes in MAP2 expression in differentiated SH-SY5Y cells in the presence of CBGA. CBGA upregulates the expression of MAP2 neuritogenesis marker. CBGA (10 μM) increased the expression of MAP2 in differentiated SH-SY5Y cells and abrogates Aβ mediated downregulation of MAP2 expression.

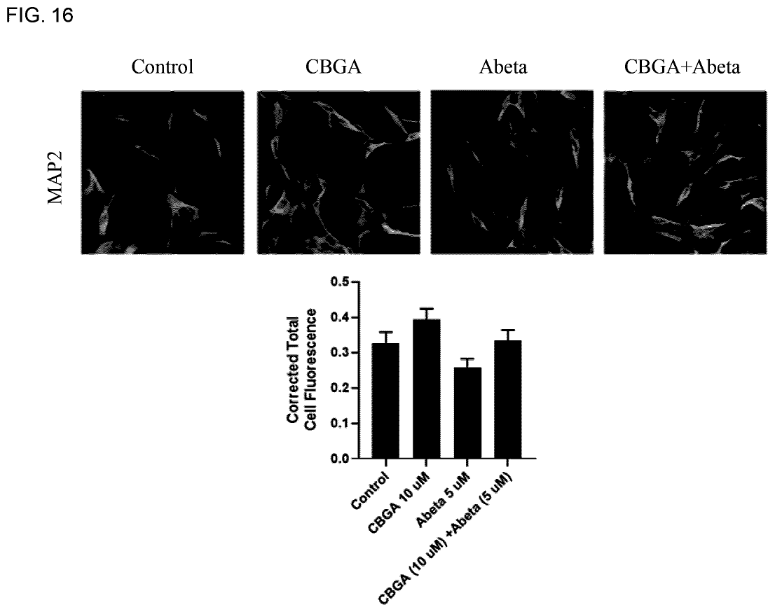

Accordingly, the effect of CBGA on the expression of MAP-2 was tested. CBGA was contacted with otherwise untreated SH-SY5Y cells or with cells previously exposed to Aβ1-42. FIG. 16 shows the results of these experiments. CBGA (10 μM) promoted upregulation of MAP-2, as detected by corrected total cell fluorescence. CBGA also resulted in elongated neurites and enhanced arborization of differentiated SH-SY5Y cells. Conversely, Aβ1-42 treatment at 5 μM downregulated MAP-2 expression and damaged neurite outgrowth. CBGA treatment of Aβ1-42 exposed cells restores MAP2 expression.

### Example 7: Study Demonstrating In Vivo CBGA Administration in AD Rodent Model

To support our hypothesis, we propose to use Alzheimer's disease in-vivo models such as APP/PS1 mice (also known as APPswe/PS1dE9; MMRRC stock #34829). These animals at age groups 3-6 months represent the stages with no plaques and NFTs formation, whereas animals aged 9-12 months displayed soluble Aβ detection, NFTs formation, memory impairment, and neurodegeneration. We plan to use both male and female mice for comparative analysis.

For the administration of cannabinoids, the cannula will be implanted stereotaxically to the lateral ventricle at coordinates [Aβ −0.2 mm, lateral: +1.3 mm, and DV: 2.2 mm of bregma] under pentobarbital anesthesia (60 mg/kg; i.p.). The cannula will be stabilized, the skin will be closed, and mice will be kept for recovery. Post-recovery, sex and age-matched genotype and their wt littermate (age group 3 months) will receive icv infusion of optimum cannabinoids several dosage level every 15 days for 3-6 months.

Experimental Readouts and Endpoints:

1. Cannabinoid infused APP/PS1 and wt mice at the ages of 6 and 9 months will be used to compare behavioural changes (learning and memory) associated with the progressive transition of AD pathogenesis.

2. To establish a functional and pathological correlation between AD and endocannabinoid system, expression of soluble and insoluble Aβ, Tau hyperphosphorylation, and CB1R/CB2R expression in cortical and hippocampal tissue extracts will be analyzed by using ELISA, immunohistochemistry and western blots in 6, 9, and 12 months old control and cannabinoid-infused APP/PS1 mice.

The inventions illustratively described herein can suitably be practiced in the absence of any element or elements, limitation or limitations not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed.

Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and sub-generic groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those skilled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description but should instead be determined with reference to the appended claims, along with the full scope of equivalents to which such claims are entitled. The disclosures of all articles and references, including patent publications, are incorporated herein by reference.

What is claimed is:

1. A method of treating a patient with a neuronal disorder, the method comprising administering a therapeutically effective amount of a pharmaceutical formulation comprising at least one cannabinoid compound, wherein the at least one cannabinoid compounds are selected from the group consisting of cannabigerolic acid (CBGA), a salt thereof, a derivative thereof, a prodrug thereof, and a combinations thereof.

2. The method of claim 1, wherein the at least one cannabinoid compound is selected from the group consisting of:
1) a compound of Formula I: 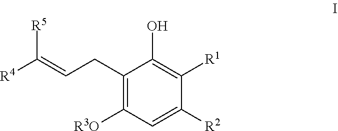
R¹ is COOH, R² is n-C₅H₁₁, R³ is H, R⁴ is (CH₂)₂CH═C(CH₃)₂ and R⁵ is Me;
R¹ is COOH, R² is n-C₅H₁₁, R³ is Me, R⁴ is (CH₂)₂CH═C(CH₃)₂ and R⁵ is Me;
R¹ is COOH, R² is n-C₃H₇, R³ is H, R⁴ is (CH₂)₂CH═C(CH₃)₂ and R⁵ is Me;
R¹ is H, R² is n-C₃H₇, R³ is H, R⁴ is (CH₂)₂CH═C(CH₃)₂ and R⁵ is Me;
R¹ is COOH, R² is n-C₅H₁₁, R³ is H, R⁴ is Me and R⁵ is (CH₂)₂CH═C(CH₃)₂;
a salt thereof, a derivative thereof, and a combination thereof;
2) a compound of Formula II: 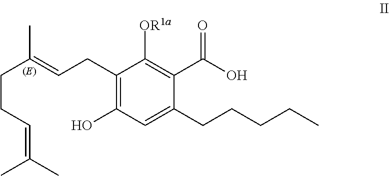
wherein R^{1a} is a prodrug moiety and salts thereof;
3) a compound of Formula II-A: 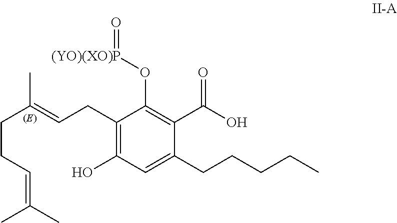
wherein X and Y can be the same or different, and are selected from the group consisting of: hydrogen, alkali metals, alkaline earth metals; and cations of pharmaceutically acceptable organic amines;
4) a compound of Formula III: 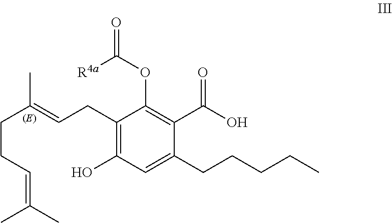
wherein R^{4a} is a straight or branched substituted or unsubstituted alkyl or R^{4a} is alkoxyalkyl, akylamine, hydroxyalkyl, or hydroxyalkylamine; and salts thereof;
5) a compound of Formula IV: 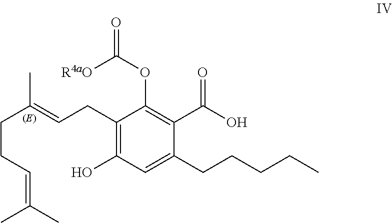
wherein R^{4a} is a straight or branched substituted or unsubstituted alkyl or R^{4a} is alkoxyalkyl, akylamine, hydroxyalkyl, or hydroxyalkylamine; and salts thereof;
6) a compound of Formula V: 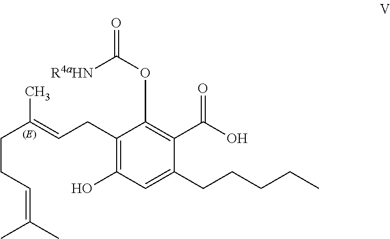
wherein R^{4a} is a straight or branched substituted or unsubstituted alkyl or R^{4a} is alkoxyalkyl, akylamine, hydroxyalkyl, or hydroxyalkylamine; and salts thereof;
7) a compound of Formula VI: 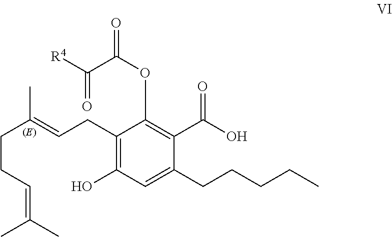
wherein R^{4a} is a straight or branched substituted or unsubstituted alkyl or R^{4a} is alkoxyalkyl, akylamine, hydroxyalkyl, or hydroxyalkylamine; and salts thereof; and
8) a compound of Formula VII: 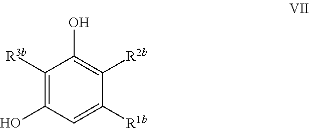
and salts thereof, wherein
R^{1b} is selected from the group consisting of C₁-C₂₀ haloalkyl, C₁-C₂₀ hydroxyalkyl, deuterated C₁-C₁₀ alkyl, tritiated C₁-C₂₀ alkyl, and C₂-C₂₀ alkenyl;
R^{2b} is selected from the group consisting of COOR^{2c} and H, preferably, COOR^{2c};
R^{2c} is selected from the group consisting of C₁-C₆ alkyl and H, preferably H; and
R^{3b} is selected from the group consisting of H and a prenyl moiety.

2. The method of claim 2, wherein the at least one cannabinoid compound is selected from Formula I, and salts thereof.

3. The method of claim 2, wherein the at least one cannabinoid compound is selected from Formula VII, and salts thereof.

4. The method of claim 1 or 2, wherein the at least one cannabinoid compound is selected from the group consisting of: 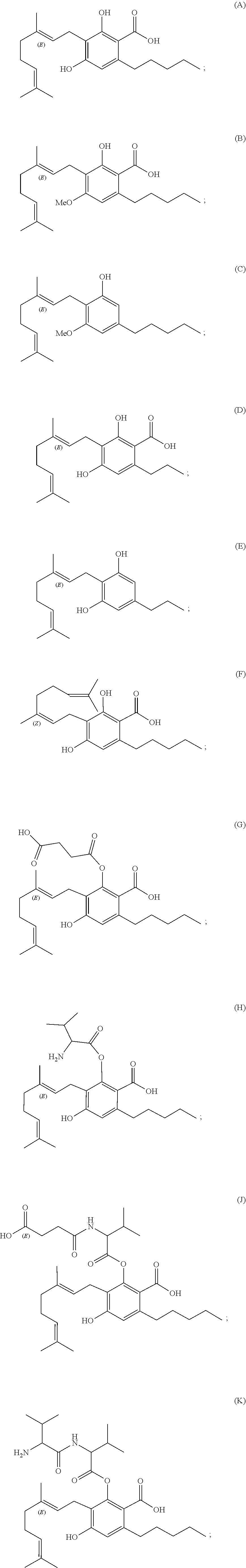 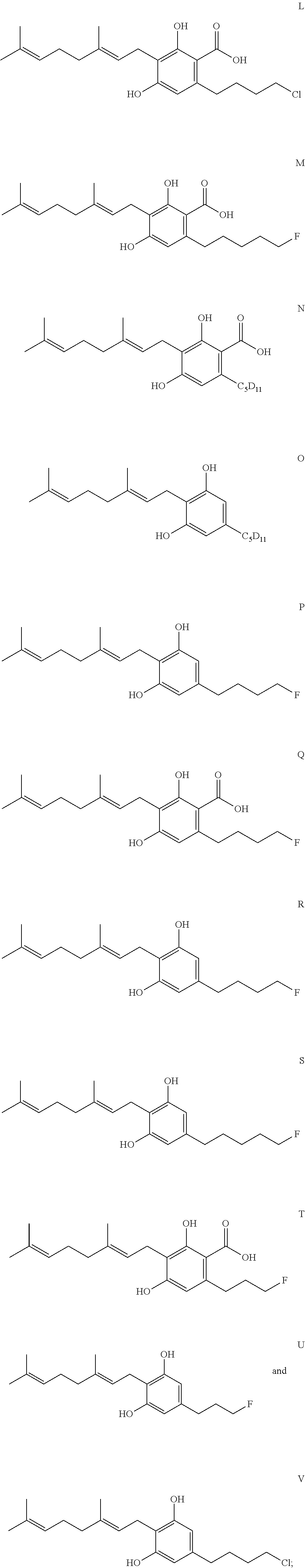
and salts thereof.

5. The method of any one of claims 2-5, wherein the at least one cannabinoid compound is not a derivative.

6. The method of any one of claims 2-6, wherein when the salt is present, it is a pharmaceutically acceptable salt.

7. The method of any one of claims 1-8, wherein the neuronal disorder is characterized by neurodegeneration.

8. The method of claim 9, wherein the neuronal disorder is a neurodegenerative disease affecting the CNS, preferably wherein the neurodegenerative disease is selected from the group consisting of Alzheimer's Disease (AD), Parkinson's Disease (PD), Amyotrophic Lateral Sclerosis (ALS), Huntington's Disease (HD) and Multiple Sclerosis (MS).

9. The method of claim 10, wherein the neurodegenerative disease is AD.

10. The method of claim 11, wherein the method comprises simultaneously or sequentially administering an additional active agent for the treatment of AD.

11. The method of any one of claims 1 to 12, wherein the therapeutically effective amount is sufficient to reduce an amount or rate of cytotoxicity of a population of affected neurons; preferably wherein the therapeutically effective amount provides a concentration of about 0.15 μM to about 20 μM of the cannabinoid compound in contact with the affected neurons, or about 0.5 μM to about 15 μM of the cannabinoid compound in contact with the affected neurons, or about 1.5 μM to about 15 μM of the cannabinoid compound in contact with the affected neurons, or about 5 μM to about 15 μM of the cannabinoid compound in contact with the affected neurons.

12. The method of any one of claims 1 to 13, wherein the pharmaceutical composition is administered by intracerebroventricular (i.c.v.) injection.

13. The method of any one of claims 1 to 8, wherein the neuronal disorder is benefited or improved by neuritogenesis.

14. The method of claim 15, wherein the neuronal disorder is selected from the group consisting of axonal injury, ischemic stroke, schizophrenia, Down syndrome, autism spectrum disorder (ASD), amyotrophic lateral sclerosis (ALS), primary lateral sclerosis, spinal muscular atrophy, motor neuron diseases, hyperacusis, presbycusis, tinnitus, chronic hearing loss, and balance disorders associated with cochlear synaptopathy and vestibular synaptopathy.

15. The method of any one of claims 1-16, wherein the cannabinoid compound is CBGA.

16. A method of promoting neurite elongation and/or restoring neurite formation in a patient in need thereof, the method comprising administering a therapeutically effective amount of a pharmaceutical formulation comprising at least one cannabinoid compound, wherein the at least one cannabinoid compounds are selected from the group consisting of CBGA, a salt thereof, a derivative thereof, a prodrug thereof, and combinations thereof.

17. The method of claim 18, wherein the at least one cannabinoid compounds are selected from the group consisting of a compound of Formula I: 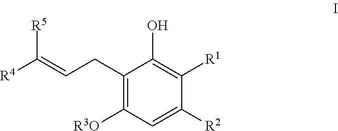
R¹ is COOH, R² is n-C₅H₁₁, R³ is H, R⁴ is (CH₂)₂CH═C(CH₃)₂ and R⁵ is Me;
R¹ is COOH, R² is n-C₅H₁₁, R³ is Me, R⁴ is (CH₂)₂CH═C(CH₃)₂ and R⁵ is Me;
R¹ is COOH, R² is n-C₃H₇, R³ is H, R⁴ is (CH₂)₂CH═C(CH₃)₂ and R⁵ is Me;
R¹ is H, R² is n-C₃H₇, R³ is H, R⁴ is (CH₂)₂CH═C(CH₃)₂ and R⁵ is Me;
R¹ is COOH, R² is n-C₅H₁₁, R³ is H, R⁴ is Me and R⁵ is (CH₂)₂CH═C(CH₃)₂;
a salt thereof, a derivative thereof, and a combination thereof;
2) a compound of Formula II: 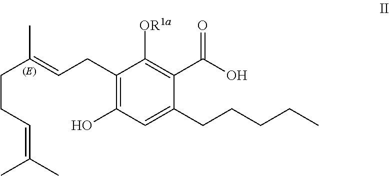
wherein R^{1a} is a prodrug moiety and salts thereof;
3) a compound of Formula II-A: 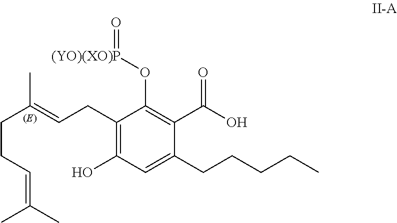
wherein X and Y can be the same or different, and are selected from the group consisting of: hydrogen, alkali metals, alkaline earth metals; and cations of pharmaceutically acceptable organic amines;
4) a compound of Formula III: 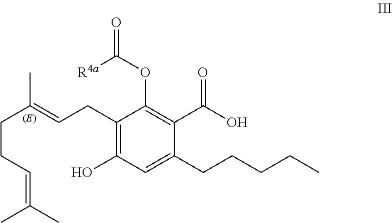
wherein R^{4a} is a straight or branched substituted or unsubstituted alkyl or R^{4a} is alkoxyalkyl, akylamine, hydroxyalkyl, or hydroxyalkylamine; and salts thereof;
5) a compound of Formula IV: 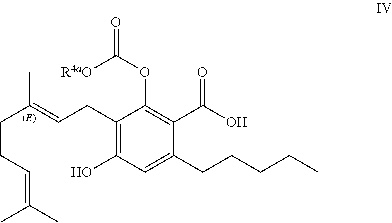
wherein R^{4a} is a straight or branched substituted or unsubstituted alkyl or R^{4a} is alkoxyalkyl, akylamine, hydroxyalkyl, or hydroxyalkylamine; and salts thereof;
6) a compound of Formula V: 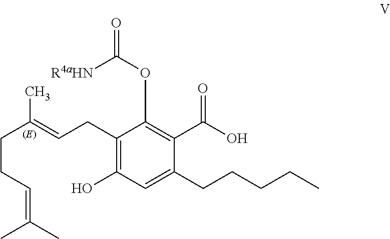
wherein R^{4a} is a straight or branched substituted or unsubstituted alkyl or R^{4a} is alkoxyalkyl, akylamine, hydroxyalkyl, or hydroxyalkylamine; and salts thereof;
7) a compound of Formula VI: 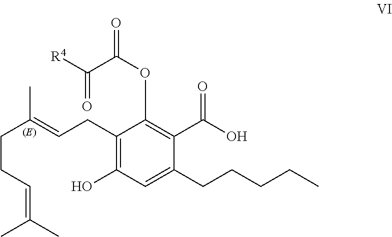
wherein R^{4a} is a straight or branched substituted or unsubstituted alkyl or R^{4a} is alkoxyalkyl, akylamine, hydroxyalkyl, or hydroxyalkylamine; and salts thereof; and
8) a compound of Formula VII: 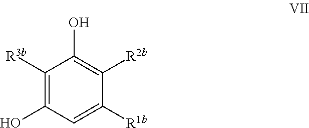
and salts thereof, wherein
R^{1b} is selected from the group consisting of C₁-C₂₀ haloalkyl, C₁-C₂₀ hydroxyalkyl, deuterated C₁-C₁₀ alkyl, tritiated C₁-C₂₀ alkyl, and C₂-C₂₀ alkenyl;
R^{2b} is selected from the group consisting of COOR^{2c} and H, preferably, COOR^{2c};
R^{2c} is selected from the group consisting of C₁-C₆ alkyl and H, preferably H; and
R^{3b} is selected from the group consisting of H and a prenyl moiety.

18. The method of claim 19, wherein the at least one cannabinoid compound is selected from Formula I, and salts thereof.

19. The method of claim 19, wherein the at least one cannabinoid compound is selected from Formula VII, and salts thereof.

20. The method of claim 18 or 19, wherein the at least one cannabinoid compound is selected from the group consisting of: 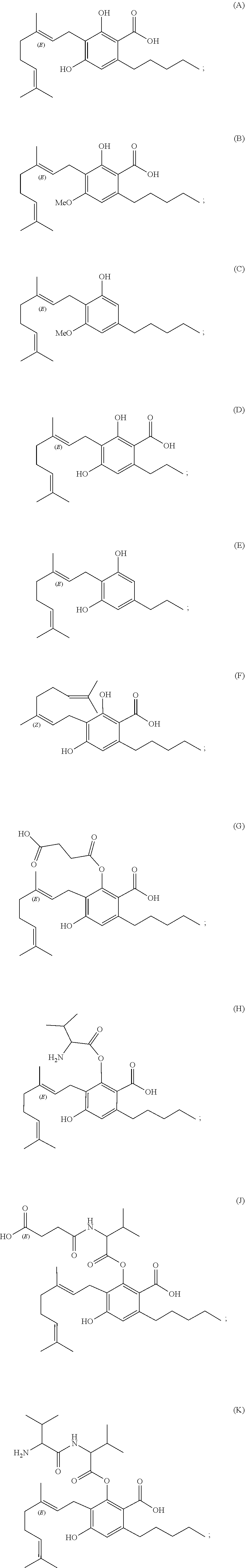 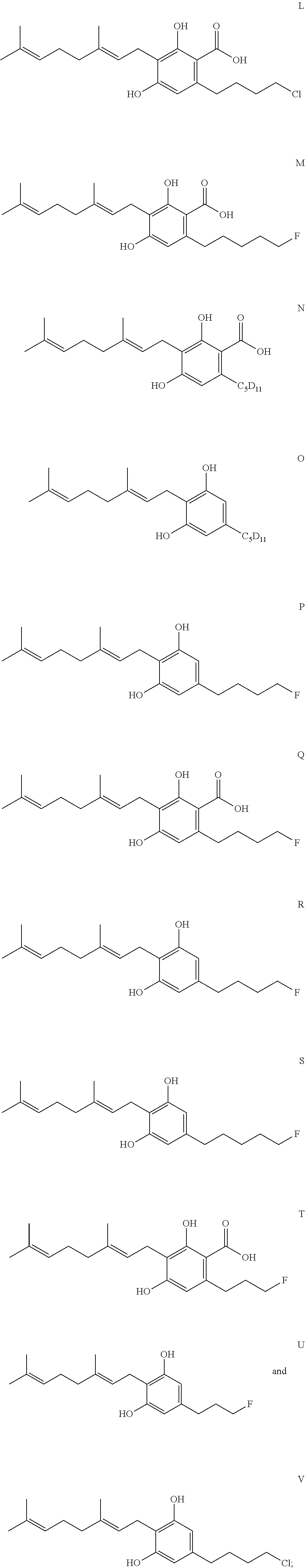
and salts thereof.

21. The method of any one of claims 19-22, wherein the at least one cannabinoid compound is not a derivative.

22. The method of any one of claims 18-23, wherein when the salt is present, it is a pharmaceutically acceptable salt.

23. The method of any one of claims 18-24, wherein the cannabinoid compound is CBGA.

24. The method of any one of claims 1 to 24, wherein the pharmaceutical composition is administered systemically.

25. The method of any one of claims 1 to 24, wherein the pharmaceutical composition is administered locally.

26. The method of any one of claims 1 to 24, wherein the pharmaceutical composition is administered weekly, daily, or twice daily.
